(19) United States
(12) Patent Application Publication
(10) Pub. No.: US 2020/0046684 A1
(43) Pub. Date: Feb. 13, 2020

(54) ANTICANCER COMBINATION THERAPY

(71) Applicant: Boehringer Ingelheim International GmbH, Ingelheim am Rhein (DE)

(72) Inventors: Maria IMPAGNATIELLO, Vienna (AT); Markus RESCHKE, Birsfelden (CH)

(21) Appl. No.: 16/497,223

(22) PCT Filed: Mar. 29, 2018
(86) PCT No.: PCT/EP2018/058106

§ 371 Date: Sep. 24, 2019

(30) Foreign Application Priority Data
Mar. 31, 2017 (EP) 17164149.1
Oct. 24, 2017 (EP) 17197931.3

(51) Int. Cl. A61K 31/4178; A61K 31/4184; A61K 31/4709; A61K 31/4725; A61P 35/00

(52) U.S. Cl. CPC A61K 31/4178; A61K 31/4184; A61K 45/06; A61K 31/4725; A61P 35/00; A61K 31/4709

(57) ABSTRACT

The invention describes anti-cancer therapies comprising using a SMAC mimetic in combination with a PD-1 antagonist, each as described herein.

The Inhibitor of Apoptosis Proteins (IAPs) are structurally characterized by the presence of at least one BIR (Baculoviral IAP Repeat) domain and consist of eight family members. Among these, XIAP, ML-IAP, cIAP1 and cIAP2 are critical regulators of cell death and survival and are attractive targets for cancer therapy. The SMAC/DIABLO protein is an endogenous antagonist of XIAP, cIAP1 and cIAP2 and intense research efforts in the last decade have resulted in the design and development of several small-molecule SMAC mimetics now in clinical trials for cancer treatment.

In addition to their role as inhibitor of apoptosis, recent findings suggest that the primary function of some IAPs consists in the regulation of inflammatory and innate immune signaling pathways. This function is attributed to their E3 ubiquitin ligase activities in the signaling cascades activated by pro-inflammatory cytokines such as TNF and by pattern recognition receptors (PRRs) such as Toll-like receptor 4 (TLR4) and the nucleotide-binding oligomerization domain 1 (NOD1) and domain 2 (NOD2) receptors [1]. The ubiquitin ligase function of cIAP proteins enables them to modulate various signaling pathways, most notably the canonical and non-canonical NF-κB signaling pathways [2]. SMAC mimetics appear to function primarily not by relieving inhibition of caspases, but rather by inducing rapid degradation of cIAP1 (by activating the auto ubiquitin ligase activity and targeting the protein to proteasome degradation), which results in altered immune signaling and sensitizes tumor cells to cell death by extrinsic death ligands from the immune system such as TNFα, TRAIL and FasL [3]. As single agent SMAC mimetics induce cell death in ˜5-15% of tumor cell lines as those cells can produce TNFα endogenously. However, this cytotoxicity can be increased to ˜50% of cancer cell lines with the addition of exogenous TNFα or TRAIL [4, 5].

TNFα binding to its receptor triggers recruitment of cIAPs via TRAF2 and TRADD to the TNFR1 and induces polyubiquitination of RIP1 that ultimately results in activation of the canonical NF-κB pathway that induces the expression of genes related to survival, proliferation or inflammation. Under conditions for which the cIAPs are absent, such as in the presence of SMAC mimetics, RIP1 is no longer ubiquitinated and forms a default death complex called the ripoptosome and in some cases (e.g. loss of caspase 8) leads to the formation of the necrosome involving RIP3. These IAP-regulated death complexes formed upon TNFα treatment can induce either caspase-8 mediated apoptosis or necroptosis, the latter being a powerful mechanism to induce immunogenic tumor cell death (ICD) and anti-tumor immunity [6, 7].

SMAC mimetics have immune modulatory function and mediate the induction of systemic cytokines (e.g. IL-6, TNFα etc.) and chemokines (e.g. MCP-1) when administered to animals or humans [8].

Cancer immunotherapy is a branch of oncology in which the immune system is used to treat cancer which is in stark contrast to existing common methods of treatment in which the tumour is directly excised or treated. This therapeutic concept is based on the identification of a number of proteins on the surface of T-cells which act to inhibit the immune function of these cells. Listed among these proteins is PD-1.

PD-1 (Programmed cell Death 1) is a cell surface receptor protein expressed on T-cells. The protein functions as an “immune checkpoint” inhibitor, i.e. it acts to modulate the activity of cells in the immune system so as to regulate and limit autoimmune diseases. It has been recently understood that many cancers can protect themselves from the immune system by modifying “immune checkpoint” inhibitors and thus avoid detection.

PD-1 has two ligands, PD-L1 and PD-L2, which interact with the cell surface receptor. On binding, PD-1 induces an intracellular signal which negatively regulates T-cell response.

As detailed above, PD-1 is a key regulator of T-cell activity. Recently, it has been shown in a range of different cancer settings that the antagonistic PD-1 antibody molecules nivolumab and pembrolizumab can be used to stimulate the immune system and thereby treat cancer.

The efficacy of therapeutic agents can be improved by using combination therapies (in particular in oncology) with other compounds and/or improving the dosage schedule. Even if the concept of combining several therapeutic agents has already been suggested, and although various combination therapies are under investigation and in clinical trials, there is still a need for new and efficient therapeutic concepts for the treatment of cancer diseases, e.g. solid tumors, which show advantages over standard therapies, such as for example better treatment outcome, beneficial effects, superior efficacy and/or improved tolerability, such as e.g. reduced side effects of the combined treatment. Specifically, there is a need for additional treatment options for patients with cancers like, e.g., lung cancer (e.g. NSCLC), breast cancer (e.g. TNBC) and multiple myeloma (MM).

It is thus an object of the present invention to provide combination treatments/methods of combination treatment providing certain advantages compared to treatments/methods of treatment currently used and/or known in the prior art. These advantages may include in vivo efficacy (e.g. improved clinical response, extend of the response, increase of the rate of response, duration of response, disease stabilization rate, duration of stabilization, time to disease progression, progression free survival (PFS) and/or overall survival (OS), later occurence of resistance and the like), safe and well tolerated administration and reduced frequency and severity of adverse events.

In this context, the inventors of the present application, surprisingly, discovered that the use of a SMAC mimetic (also called IAP inhibitor) in combination with a PD-1 (Programmed cell Death 1) antagonist, i.e. an anti-PD-1 or anti-PD-L1 antibody in the context of the invention, has the potential to improve clinical outcome compared to the use of either a SMAC mimetic or a PD-1 antagonist alone.

Thus, the invention relates to methods for the treatment and/or prevention of oncological or hyperproliferative diseases, in particular cancer, comprising the combined administration of a SMAC mimetic and a PD-1 antagonist, each as described herein, as well as to medical uses, to uses, to pharmaceutical compositions or combinations and kits comprising such therapeutic agents.

Further, the invention relates to anti-cancer therapies comprising using a SMAC mimetic and a PD-1 antagonist, each as described herein, in combination.

For the treatment of diseases of oncological nature, a large number of anticancer agents (including target-specific and non-target-specific anticancer agents) have already been suggested, which can be used as monotherapy or as combination therapy involving more than one agent (e.g. dual or triple combination therapy) and/or which may be combined with radiotherapy (e.g. irradiation treatment), radio-immunotherapy and/or surgery.

It is a purpose of the present invention to provide combination therapies with the therapeutic agents described herein for treating or controlling various malignancies (e.g. based on cooperative, complementary, interactive or improving effects of the active components involved in combination).

Thus, in one aspect the invention provides a method of treating and/or preventing an oncological or hyperproliferative disease, in particular cancer, comprising administering to a patient in need thereof a therapeutically effective amount of a SMAC mimetic and a therapeutically effective amount of a PD-1 antagonist, each as described herein.

In another aspect the method of treating and/or preventing further comprises administering a therapeutically effective amount of one or more additional therapeutic agent(s) as described herein.

Such a combined treatment may be given as a non-fixed (e.g. free) combination of the substances or in the form of a fixed combination, including kit-of-parts.

In another aspect the invention provides a combination of a SMAC mimetic and a PD-1 antagonist, each as described herein, particularly for use in a method of treating and/or preventing an oncological or hyperproliferative disease, in particular cancer, as described herein, said method comprising administering to a patient in need thereof a therapeutically effective amount of the combination.

In another aspect the combination further comprises one or more additional therapeutic agent(s) as described herein.

In another aspect the invention refers to a SMAC mimetic as described herein for use in a method of treating and/or preventing an oncological or hyperproliferative disease, in particular cancer, as described herein, said method comprising administering the SMAC mimetic in combination with a PD-1 antagonist as described herein to a patient in need thereof.

In another aspect the method of treating and/or preventing further comprises administering in combination with one or more additional therapeutic agent(s) as described herein.

In another aspect the invention refers to a PD-1 antagonist as described herein for use in a method of treating and/or preventing an oncological or hyperproliferative disease, in particular cancer, as described herein, said method comprising administering the PD-1 antagonist in combination with a SMAC mimetic as described herein to a patient in need thereof.

In another aspect the method of treating and/or preventing further comprises administering in combination with one or more additional therapeutic agent(s) as described herein.

In another aspect the invention refers to a kit comprising
a first pharmaceutical composition or dosage form comprising a SMAC mimetic as described herein, and, optionally, one or more pharmaceutically acceptable carriers, excipients and/or vehicles, and
a second pharmaceutical composition or dosage form comprising a PD-1 antagonist as described herein, and, optionally, one or more pharmaceutically acceptable carriers, excipients and/or vehicles.

In another aspect the kit comprises one or more additional pharmaceutical composition(s) or dosage form(s) each comprising one additional therapeutic agent as described herein, and, optionally, one or more pharmaceutically acceptable carriers, excipients and/or vehicles.

In another aspect the invention refers to the aforementioned kits further comprising
a package insert comprising printed instructions for simultaneous, concurrent, sequential, successive, alternate or separate use in the treatment and/or prevention of an oncological or hyperproliferative disease, in particular cancer, as described herein, in a patient in need thereof.

In another aspect the invention refers to the aforementioned kits for use in a method of treating and/or preventing an oncological or hyperproliferative disease, in particular cancer, as described herein.

In another aspect the invention refers to a pharmaceutical composition comprising
a SMAC mimetic as described herein,
a PD-1 antagonist as described herein, and,
optionally, one or more pharmaceutically acceptable carriers, excipients and/or vehicles.

In another aspect the pharmaceutical composition comprises one or more additional therapeutic agent(s) as described herein.

In another aspect the invention refers to the use of a SMAC mimetic as described herein for preparing a pharmaceutical composition for use in a method of treating and/or preventing an oncological or hyperproliferative disease, in particular cancer, as described herein, wherein the SMAC mimetic is to be used in combination with a PD-1 antagonist as described herein.

In another aspect of the use of the SMAC mimetic the SMAC mimetic is to be used in combination with a PD-1 antagonist as described herein and one or more additional therapeutic agent(s) as described herein.

In another aspect the invention refers to the use of a PD-1 antagonist as described herein for preparing a pharmaceutical composition for use in a method of treating and/or preventing an oncological or hyperproliferative disease, in particular cancer, as described herein, wherein the PD-1 antagonist is to be used in combination with a SMAC mimetic as described herein.

In another aspect of the use of the PD-1 antagonist the PD-1 antagonist is to be used in combination with a SMAC mimetic as described herein and one or more additional therapeutic agent(s) as described herein.

In another aspect the invention refers to the use of a SMAC mimetic and a PD-1 antagonist, each as described herein, for preparing a pharmaceutical composition for use in a method of treating and/or preventing an oncological or hyperproliferative disease, in particular cancer., as described herein.

In another aspect, the invention refers to the use of a SMAC mimetic, a PD-1 antagonist and one or more additional therapeutic agent(s), each as described herein, for preparing a pharmaceutical composition for use in a method of treating and/or preventing an oncological or hyperproliferative disease, in particular cancer, as described herein.

In another aspect, the invention refers to a combination, a pharmaceutical composition or a kit according to the invention, each as described herein, comprising, consisting or consisting essentially of a SMAC mimetic and a PD-1 antagonist, each as described herein, for use in a method of treating and/or preventing an oncological or hyperproliferative disease, in particular cancer, as described herein.

### BRIEF DESCRIPTION OF THE FIGURES

FIG. 1 shows the anti-tumor activity of the exemplary SMAC mimetic BIA-1 as single agent and in combination with RMP1-14, a mouse tool antibody to PD-1, in a subcutaneous syngeneic mouse model derived from the breast cancer cell line EMT6 in Balb/c mice.

FIG. 2 shows the anti-tumor activity of the exemplary SMAC mimetics BIA-1 and BIA-2 as single agents and in combination with RMP1-14, a mouse tool antibody to PD-1, in a subcutaneous syngeneic mouse model derived from the bladder cancer cell line MBT-2 in C3H mice.

FIG. 3 shows the anti-tumor activity of the exemplary SMAC mimetics BIA-1 and BIA-2 as single agents and in combination with RMP1-14, a mouse tool antibody to PD-1, in the Vk12598 multiple myeloma transplantable model in C57BL/6J mice.

FIG. 4 shows the potentiating activity of the exemplary SMAC mimetic BIA-1 on the stimulation of antigen-specific T cell response by the anti-PD1 MK3465

### SMAC MIMETIC

The SMAC mimetic within the meaning of this invention and all its embodiments is a compound which binds to IAP proteins and induces their degradation.

Preferably, the SMAC mimetic within this invention and all its embodiments is selected from the group consisting of the following (A0):
a SMAC mimetic (i.e. a compound) as (generically and/or specifically) disclosed in WO 2013/127729, or a pharmaceutically acceptable salt thereof;
a SMAC mimetic (i.e. a compound) as (generically and/or specifically) disclosed in WO 2015/025018, or a pharmaceutically acceptable salt thereof;
a SMAC mimetic (i.e. a compound) as (generically and/or specifically) disclosed in WO 2015/025019, or a pharmaceutically acceptable salt thereof;
a SMAC mimetic (i.e. a compound) as (generically and/or specifically) disclosed in WO 2016/023858, or a pharmaceutically acceptable salt thereof;
a SMAC mimetic (i.e. a compound) as (generically and/or specifically) disclosed in WO 2008/0016893, or a pharmaceutically acceptable salt thereof;
LCL161, i.e. compound A in example 1 of WO 2008/016893 (page 28/29; [122]), or a pharmaceutically acceptable salt thereof;
the SMAC mimetic known as Debio-1143, or a pharmaceutically acceptable salt thereof;
the SMAC mimetic known as birinapant, or a pharmaceutically acceptable salt thereof;
the SMAC mimetic known as ASTX-660, or a pharmaceutically acceptable salt thereof;
the SMAC mimetic known as CUDC-427, or a pharmaceutically acceptable salt thereof
any one of the SMAC mimetics 1 to 26 in table 1 or a pharmaceutically acceptable salt thereof:

TABLE 1

| | |
|---|---|
| 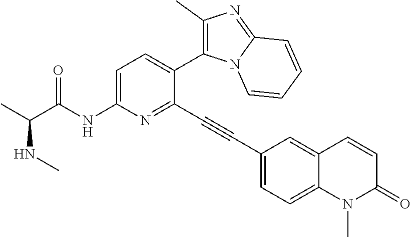 [SMILES: CN[C@@H](C)C(=O)Nc1ccc(-c2c(C)nc3ccccn23)c(C#Cc2ccc3c(ccc(=O)n3C)c2)n1] | 1 |
| 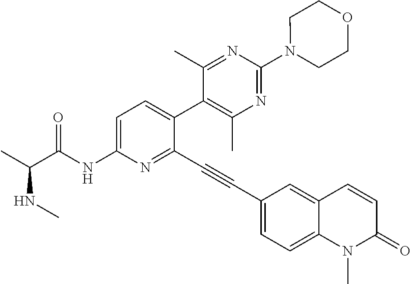 [SMILES: CN[C@@H](C)C(=O)Nc1ccc(-c2c(C)nc(N3CCOCC3)nc2C)c(C#Cc2ccc3c(ccc(=O)n3C)c2)n1] | 2 |
| 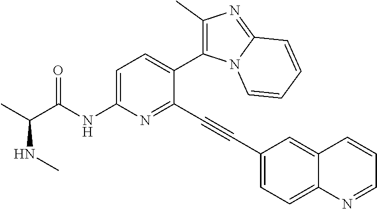 [SMILES: CN[C@@H](C)C(=O)Nc1ccc(-c2c(C)nc3ccccn23)c(C#Cc2ccc3ncccc3c2)n1] | 3 |
| 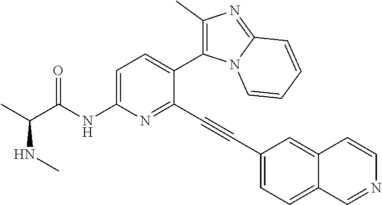 [SMILES: CN[C@@H](C)C(=O)Nc1ccc(-c2c(C)nc3ccccn23)c(C#Cc2ccc3cnccc3c2)n1] | 4 |
| 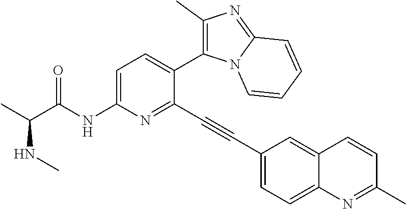 [SMILES: CN[C@@H](C)C(=O)Nc1ccc(-c2c(C)nc3ccccn23)c(C#Cc2ccc3nc(C)ccc3c2)n1] | 5 |
| 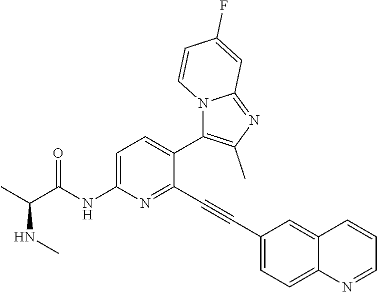 [SMILES: CN[C@@H](C)C(=O)Nc1ccc(-c2c(C)nc3cc(F)ccn23)c(C#Cc2ccc3ncccc3c2)n1] | 6 |
| 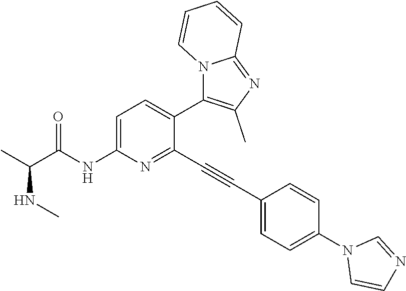 [SMILES: CN[C@@H](C)C(=O)Nc1ccc(-c2c(C)nc3ccccn23)c(C#Cc2ccc(-n3ccnc3)cc2)n1] | 7 |
| 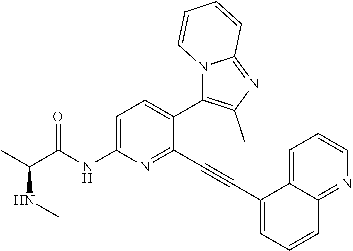 [SMILES: CN[C@@H](C)C(=O)Nc1ccc(-c2c(C)nc3ccccn23)c(C#Cc2cccc3ncccc23)n1] | 8 |
| 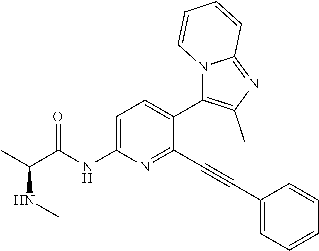 [SMILES: CN[C@@H](C)C(=O)Nc1ccc(-c2c(C)nc3ccccn23)c(C#Cc2ccccc2)n1] | 9 |
| 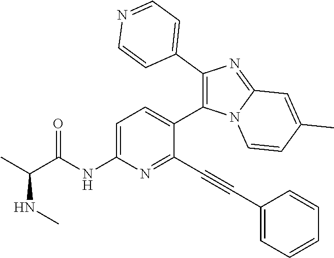 [SMILES: CN[C@@H](C)C(=O)Nc1ccc(-c2c(-c3ccncc3)nc3cc(C)ccn23)c(C#Cc2ccccc2)n1] | 10 |
| 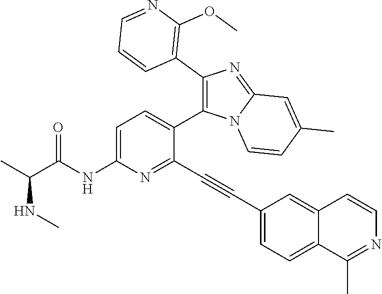 [SMILES: CN[C@@H](C)C(=O)Nc1ccc(-c2c(-c3cccnc3OC)nc3cc(C)ccn23)c(C#Cc2ccc3c(C)nccc3c2)n1] | 11 |
| 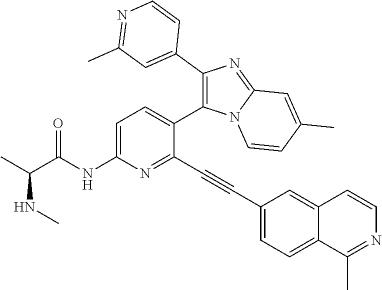 [SMILES: CN[C@@H](C)C(=O)Nc1ccc(-c2c(-c3ccnc(C)c3)nc3cc(C)ccn23)c(C#Cc2ccc3c(C)nccc3c2)n1] | 12 |
| 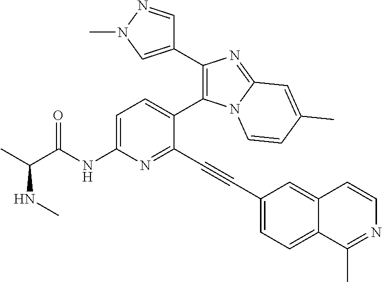 [SMILES: CN[C@@H](C)C(=O)Nc1ccc(-c2c(-c3cnn(C)c3)nc3cc(C)ccn23)c(C#Cc2ccc3c(C)nccc3c2)n1] | 13 |
| 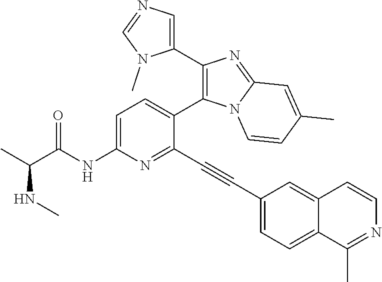 [SMILES: CN[C@@H](C)C(=O)Nc1ccc(-c2c(-c3cncn3C)nc3cc(C)ccn23)c(C#Cc2ccc3c(C)nccc3c2)n1] | 14 |
| 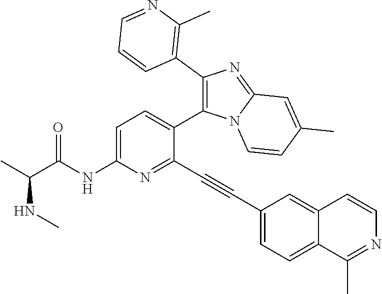 [SMILES: CN[C@@H](C)C(=O)Nc1ccc(-c2c(-c3cccnc3C)nc3cc(C)ccn23)c(C#Cc2ccc3c(C)nccc3c2)n1] | 15 |
| 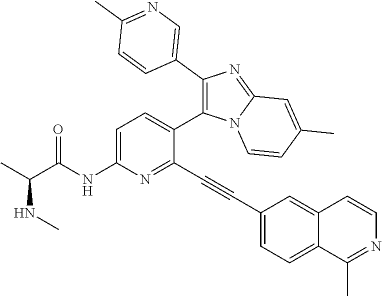 [SMILES: CN[C@@H](C)C(=O)Nc1ccc(-c2c(-c3ccc(C)nc3)nc3cc(C)ccn23)c(C#Cc2ccc3c(C)nccc3c2)n1] | 16 |
| 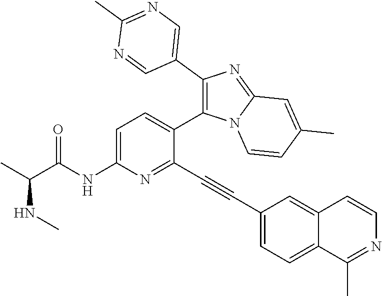 [SMILES: CN[C@@H](C)C(=O)Nc1ccc(-c2c(-c3cnc(C)nc3)nc3cc(C)ccn23)c(C#Cc2ccc3c(C)nccc3c2)n1] | 17 |
| 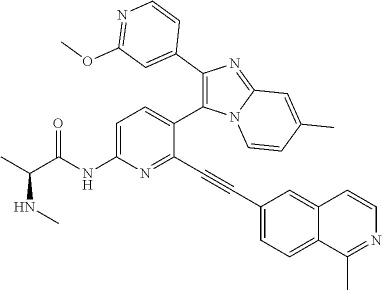 [SMILES: CN[C@@H](C)C(=O)Nc1ccc(-c2c(-c3ccnc(OC)c3)nc3cc(C)ccn23)c(C#Cc2ccc3c(C)nccc3c2)n1] | 18 |
| 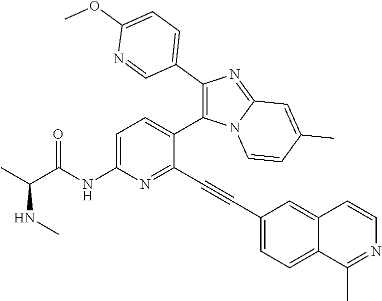 [SMILES: CN[C@@H](C)C(=O)Nc1ccc(-c2c(-c3ccc(OC)nc3)nc3cc(C)ccn23)c(C#Cc2ccc3c(C)nccc3c2)n1] | 19 |
| 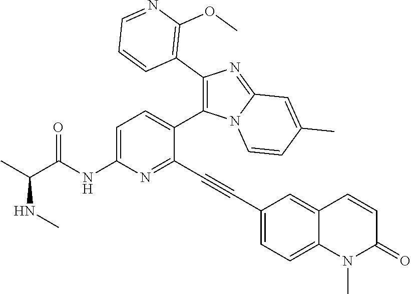 [SMILES: CN[C@@H](C)C(=O)Nc1ccc(-c2c(-c3cccnc3OC)nc3cc(C)ccn23)c(C#Cc2ccc3c(ccc(=O)n3C)c2)n1] | 20 |
| 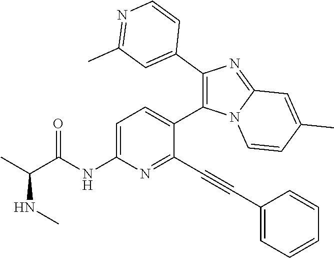 [SMILES: CN[C@@H](C)C(=O)Nc1ccc(-c2c(-c3ccnc(C)c3)nc3cc(C)ccn23)c(C#Cc2ccccc2)n1] | 21 |
| 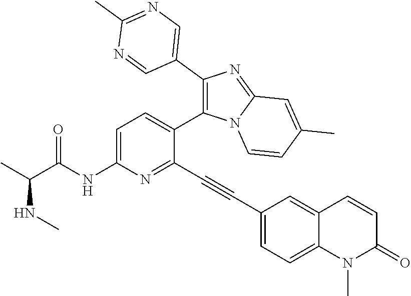 [SMILES: CN[C@@H](C)C(=O)Nc1ccc(-c2c(-c3cnc(C)nc3)nc3cc(C)ccn23)c(C#Cc2ccc3c(ccc(=O)n3C)c2)n1] | 22 |
| 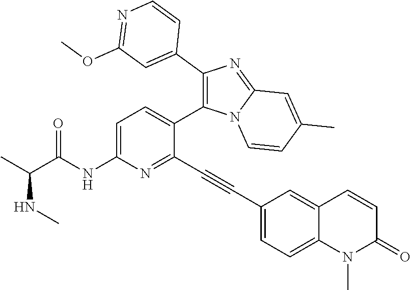 [SMILES: CN[C@@H](C)C(=O)Nc1ccc(-c2c(-c3ccnc(OC)c3)nc3cc(C)ccn23)c(C#Cc2ccc3c(ccc(=O)n3C)c2)n1] | 23 |
| 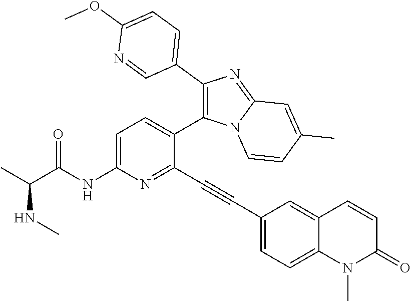 [SMILES: CN[C@@H](C)C(=O)Nc1ccc(-c2c(-c3ccc(OC)nc3)nc3cc(C)ccn23)c(C#Cc2ccc3c(ccc(=O)n3C)c2)n1] | 24 |
| 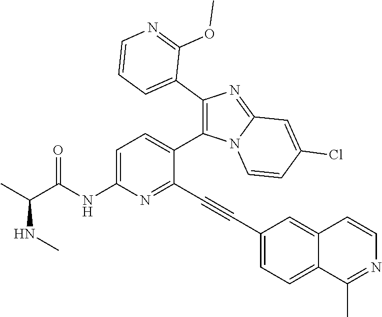 [SMILES: CN[C@@H](C)C(=O)Nc1ccc(-c2c(-c3cccnc3OC)nc3cc(Cl)ccn23)c(C#Cc2ccc3c(C)nccc3c2)n1] | 25 |
| 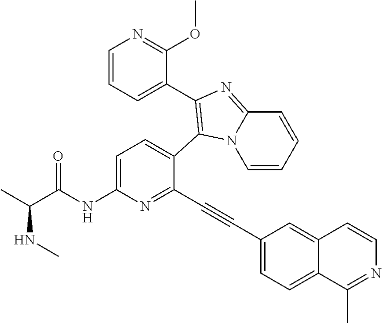 [SMILES: CN[C@@H](C)C(=O)Nc1ccc(-c2c(-c3cccnc3OC)nc3ccccn23)c(C#Cc2ccc3c(C)nccc3c2)n1] | 26 |

Example compounds 1 to 10 in Table 1 are disclosed in WO 2013/127729. Example compounds 11 to 26 in Table 1 are disclosed in WO 2016/023858.

The term “SMAC mimetic” as used herein also includes the SMAC mimetics listed above in the form of a tautomer, of a pharmaceutically acceptable salt, of a hydrate or of a solvate (including a hydrate or solvate of a pharmaceutically acceptable salt). It also includes the SMAC mimetic in all its solid, preferably crystalline, forms and in all the crystalline forms of its pharmaceutically acceptable salts, hydrates and solvates (including hydrates and solvates of pharmaceutically acceptable salts).

All SMAC mimetics listed above are known in the art with the respective synthesis and properties. All patent applications referred to above are incorporated by reference in their entirety.

In one embodiment the SMAC mimetic is LCL161 or a pharmaceutically acceptable salt thereof (A1).

In another embodiment the SMAC mimetic is compound 1 in table 1 or a pharmaceutically acceptable salt thereof (A2).

In another embodiment the SMAC mimetic is compound 2 in table 1 or a pharmaceutically acceptable salt thereof (A3).

In another embodiment the SMAC mimetic is compound 3 in table 1 or a pharmaceutically acceptable salt thereof (A4).

In another embodiment the SMAC mimetic is compound 4 in table 1 or a pharmaceutically acceptable salt thereof (A5).

In another embodiment the SMAC mimetic is compound 5 in table 1 or a pharmaceutically acceptable salt thereof (A6).

In another embodiment the SMAC mimetic is compound 6 in table 1 or a pharmaceutically acceptable salt thereof (A7).

In another embodiment the SMAC mimetic is compound 7 in table 1 or a pharmaceutically acceptable salt thereof (A8).

In another embodiment the SMAC mimetic is compound 8 in table 1 or a pharmaceutically acceptable salt thereof (A9).

In another embodiment the SMAC mimetic is compound 9 in table 1 or a pharmaceutically acceptable salt thereof (A10).

In another embodiment the SMAC mimetic is compound 10 in table 1 or a pharmaceutically acceptable salt thereof (A11).

In another embodiment the SMAC mimetic is compound 11 in table 1 or a pharmaceutically acceptable salt thereof (A12).

In another embodiment the SMAC mimetic is compound 12 in table 1 or a pharmaceutically acceptable salt thereof (A13).

In another embodiment the SMAC mimetic is compound 13 in table 1 or a pharmaceutically acceptable salt thereof (A14).

In another embodiment the SMAC mimetic is compound 14 in table 1 or a pharmaceutically acceptable salt thereof (A15).

In another embodiment the SMAC mimetic is compound 15 in table 1 or a pharmaceutically acceptable salt thereof (A16).

In another embodiment the SMAC mimetic is compound 16 in table 1 or a pharmaceutically acceptable salt thereof (A17).

In another embodiment the SMAC mimetic is compound 17 in table 1 or a pharmaceutically acceptable salt thereof (A18).

In another embodiment the SMAC mimetic is compound 18 in table 1 or a pharmaceutically acceptable salt thereof (A19).

In another embodiment the SMAC mimetic is compound 19 in table 1 or a pharmaceutically acceptable salt thereof (A20).

In another embodiment the SMAC mimetic is compound 20 in table 1 or a pharmaceutically acceptable salt thereof (A21).

In another embodiment the SMAC mimetic is compound 21 in table 1 or a pharmaceutically acceptable salt thereof (A22).

In another embodiment the SMAC mimetic is compound 22 in table 1 or a pharmaceutically acceptable salt thereof (A23).

In another embodiment the SMAC mimetic is compound 23 in table 1 or a pharmaceutically acceptable salt thereof (A24).

In another embodiment the SMAC mimetic is compound 24 in table 1 or a pharmaceutically acceptable salt thereof (A25).

In another embodiment the SMAC mimetic is compound 25 in table 1 or a pharmaceutically acceptable salt thereof (A26).

In another embodiment the SMAC mimetic is compound 26 in table 1 or a pharmaceutically acceptable salt thereof (A27).

All embodiments (A1) to (A27) are preferred embodiments of embodiment (A0) in respect of the nature of the SMAC mimetic.

To be used in therapy, the SMAC mimetic is included into pharmaceutical compositions appropriate to facilitate administration to animals or humans.

Typical pharmaceutical compositions for administering the SMAC mimetic of the invention include for example tablets, capsules, suppositories, solutions, e.g. solutions for injection (s.c., i.v., i.m.) and infusion, elixirs, emulsions or dispersible powders. The content of the pharmaceutically active compound(s) may be in the range from 0.1 to 90 wt.-%, preferably 40 to 60 wt.-% of the composition as a whole, e.g. in amounts which are sufficient to achieve the desired dosage range. The single dosages may, if necessary, be given several times a day to deliver the desired total daily dose.

Typical tablets may be obtained, for example, by mixing the active substance(s), optionally in combination, with known excipients, for example inert diluents such as calcium carbonate, calcium phosphate, cellulose or lactose, disintegrants such as corn starch or alginic acid or crospovidon, binders such as starch or gelatine, lubricants such as magnesium stearate or talc and/or agents for delaying release, such as carboxymethyl cellulose, cellulose acetate phthalate, or polyvinyl acetate. The tablets may be prepared by usual processes, such as e.g. by direct compression or roller compaction. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number of layers to achieve delayed release, possibly using the excipients mentioned above for the tablets.

Syrups or elixirs containing the active substance(s) may additionally contain a sweetener such as saccharine, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection and infusion are prepared in the usual way, e.g. with the addition of isotonic agents, preservatives such as p-hydroxybenzoates, or stabilisers such as alkali metal salts of ethylenediamine tetraacetic acid, optionally using emulsifiers and/or dispersants, whilst if water is used as the diluent, for example, organic solvents may optionally be used as solvating agents or dissolving aids, and transferred into injection vials or ampoules or infusion bottles.

Capsules containing the active substance(s) may for example be prepared by mixing the active substance(s) with inert carriers such as lactose or sorbitol and packing them into gelatine capsules.

Typical suppositories may be made for example by mixing the active substance(s) with carriers provided for this purpose, such as neutral fats or polyethyleneglycol or the derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose) emulsifiers (e.g. lignin, spent sulphite liquors, methylcellulose, starch and polyvinylpyrrolidone) and lubricants (e.g. magnesium stearate, talc, stearic acid and sodium lauryl sulphate).

The SMAC mimetic of this invention and all its embodiments is administered by the usual methods, preferably by oral or parenteral route, most preferably by oral route. For oral administration the tablets may contain, apart from the abovementioned carriers, additives such as sodium citrate, calcium carbonate and dicalcium phosphate together with various additives such as starch, preferably potato starch, gelatine and the like. Moreover, lubricants such as magnesium stearate, sodium lauryl sulphate and talc may be used at the same time for the tabletting process. In the case of aqueous suspensions the active substances may be combined with various flavour enhancers or colourings in addition to the excipients mentioned above.

For parenteral use, solutions of the active substances with suitable liquid carriers may be used.

For LCL161 the dosage for oral use and administration schedule is, e.g., as disclosed in WO 2016/054555, page 14, first paragraph, and page 126/127.

The dosage for oral use for SMAC mimetics in table 1 is from 1 mg to 2000 mg per day (e.g. 100 mg to 1000 mg per day; in a more preferred embodiment from 200 mg to 400 mg per day; most preferred is 300 mg per day). All amounts given refer to the free base of the SMAC mimetic in table 1 and may be proportionally higher if a pharmaceutically acceptable salt or other solid form is used.

Preferably, the SMAC mimetic is dosed once daily (q.d.).

The dosage for intravenous use is from 1 mg to 1000 mg per hour, preferably between 5 and 500 mg per hour.

However, it may sometimes be necessary to depart from the amounts specified, depending on the body weight, the route of administration, the individual response to the drug, the nature of its formulation and the time or interval over which the drug is administered. Thus, in some cases it may be sufficient to use less than the minimum dose given above, whereas in other cases the upper limit may have to be exceeded. When administering large amounts it may be advisable to divide them up into a number of smaller doses spread over the day.

PD-1 Antagonist

A PD-1 antagonist within the meaning of this invention and all of its embodiments is a compound that inhibits the interaction of PD-1 with its receptor(s) or ligand(s), Preferably, the PD-1 antagonist is an inhibitor of PD-1 or an inhibitor of PD-L1, more preferably an anti-PD-1 antibody or an anti-PD-L1 antibody, most preferably a humanized or fully human anti-PD-1 antibody or a humanized or fully human anti-PD-L1 antibody.

The term “antibody” encompasses antibodies, antibody fragments, antibody-like molecules and conjugates with any of the above. Antibodies include, but are not limited to, poly- or monoclonal, chimeric, humanized, human, mono-, bi- or multispecific antibodies. The term “antibody” shall encompass complete immunoglobulins as they are produced by lymphocytes and for example present in blood sera, monoclonal antibodies secreted by hybridoma cell lines, polypeptides produced by recombinant expression in host cells, which have the binding specificity of immunoglobulins or monoclonal antibodies, and molecules which have been derived from such immunoglobulins, monoclonal antibodies, or polypeptides by further processing while retaining their binding specificity. In particular, the term “antibody” includes complete immunoglobulins comprising two heavy chains and two light chains. In another embodiment, the term encompasses a fragment of an immunoglobulin, like Fab fragments. In another embodiment, the term “antibody” encompasses a polypeptide having one or more variable domains derived from an immunobulin, like single chain antibodies (scFv), single domain antibodies, and the like.

PD-1 antagonists are well-known in the art, e.g. reviewed by Li et al., Int. J. Mol. Sci. 2016, 17, 1151 (incorporated herein by reference). Any PD-1 antagonist, especially antibodies, such as those disclosed by Li et al. as well as the further antibodies disclosed herein below, can be used according to the invention.

Most preferred, the PD-1 antagonist within this invention and all its embodiments is selected from the group consisting of the following (B0):
pembrolizumab (anti-PD-1 antibody);
nivolumab (anti-PD-1 antibody);
pidilizumab (anti-PD-1 antibody);
PDR-001 (anti-PD-1 antibody)
Atezolizumab (anti-PD-L1 antibody);
Avelumab (anti-PD-L1 antibody);
Durvalumab (anti-PD-L1 antibody);
an anti-PD-1 antibody (generically and/or specifically) disclosed in WO 2015/112900:
   any one of the antibodies as defined in table 1 in WO 2015/112900 (page 171)
   any one of the humanized antibodies as defined in table 1 in WO 2015/112900 (page 171)
   any one of BAP049-hum01 to BAP049-hum16 as defined in table 1 in WO 2015/112900 (page 171)
   any one of BAP049-Clone-A to BAP049-Clone-E as defined in table 1 in WO 2015/112900 (page 171)
an anti-PD-L1 antibody (generically and/or specifically) disclosed in WO 2016/061142:
   any one of the antibodies as defined in table 1 in WO 2016/061142 (page 265);
   any one of the humanized antibodies as defined in table 1 in WO 2016/061142 (page 265);
   any one of BAP058-hum01 to BAP058-hum17 as defined in table 1 in WO 2016/061142 (page 265)
   any one of BAP058-Clone-K to BAP058-Clone-0 as defined in table 1 in WO 2016/061142 (page 265)
PD1-1, PD1-2, PD1-3, PD1-4, and PD1-5 as disclosed herein below (anti-PD-1 antibodies)

Pembrolizumab (formerly also known as lambrolizumab; trade name Keytruda; also known as MK-3475) disclosed e.g. in Hamid, O. et al. (2013) New England Journal of Medicine 369(2):134-44, is a humanized IgG4 monoclonal antibody that binds to PD-1; it contains a mutation at C228P designed to prevent Fc-mediated cytotoxicity. Pembrolizumab is e.g. disclosed in U.S. Pat. No. 8,354,509 and WO 2009/114335. It is approved by the FDA for the treatment of patients suffering from unresectable or metastatic melanoma and patients with metastatic NSCLC.

Nivolumab (CAS Registry Number: 946414-94-4; BMS-936558 or MDX1106b) is a fully human IgG4 monoclonal antibody which specifically blocks PD-1, lacking detectable antibody-dependent cellular toxicity (ADCC). Nivolumab is e.g. disclosed in U.S. Pat. No. 8,008,449 and WO 2006/121168. It has been approved by the FDA for the treatment of patients suffering from unresectable or metastatic melanoma, metastatic NSCLC and advanced renal cell carcinoma.

Pidilizumab (CT-011; Cure Tech) is a humanized IgG1k monoclonal antibody that binds to PD-1. Pidilizumab is e.g. disclosed in WO 2009/101611.

PDR-001 or PDR001 is a high-affinity, ligand-blocking, humanized anti-PD-1 IgG4 antibody that blocks the binding of PD-L1 and PD-L2 to PD-1. PDR-001 is disclosed in WO 2015/112900 and WO 2017/019896.

Antibodies PD1-1 to PD1-5 are antibody molecules defined by the sequences as shown in Table 2, wherein HC denotes the (full length) heavy chain and LC denotes the (full length) light chain:

TABLE 2

| SEQ | Sequence | |
|---|---|---|
| ID NO: | name | Amino acid sequence |
| 1 | HC of | EVMLVESGGGLVQPGGSLRLSCTASGFTFS |
| | PD1-1 | ASAMSWVRQAPGKGLEWVAYISGGGGDTYY |
| | | SSSVKGRFTISRDNAKNSLYLQMNSLRAED |
| | | TAVYYCARHSNVNYYAMDYWGQGTLVTVSS |
| | | ASTKGPSVFPLAPCSRSTSESTAALGCLVK |
| | | DYFPEPVTVSWNSGALTSGVHTFPAVLQSS |
| | | GLYSLSSVVTVPSSSLGTKTYTCNVDHKPS |
| | | NTKVDKRVESKYGPPCPPCPAPEFLGGPSV |
| | | FLFPPKPKDTLMISRTPEVTCVVVDVSQED |
| | | PEVQFNWYVDGVEVHNAKTKPREEQFNSTY |
| | | RVVSVLTVLHQDWLNGKEYKCKVSNKGLPS |
| | | SIEKTISKAKGQPREPQVYTLPPSQEEMTK |
| | | NQVSLTCLVKGFYPSDIAVEWESNGQPENN |
| | | YKTTPPVLDSDGSFFLYSRLTVDKSRWQEG |
| | | NVFSCSVMHEALHNHYTQKSLSLSLG |
| 2 | LC of | EIVLTQSPATLSLSPGERATMSCRASENID |
| | PD1-1 | TSGISFMNWYQQKPGQAPKLLIYVASNQGS |
| | | GIPARFSGSGSGTDFTLTISRLEPEDFAVY |
| | | YCQQSKEVPWTFGQGTKLEIKRTVAAPSVF |
| | | IFPPSDEQLKSGTASVVCLLNNFYPREAKV |
| | | QWKVDNALQSGNSQESVTEQDSKDSTYSLS |
| | | STLTLSKADYEKHKVYACEVTHQGLSSPVT |
| | | KSFNRGEC |
| 3 | HC of | EVMLVESGGGLVQPGGSLRLSCTASGFTFS |
| | PD1-2 | ASAMSWVRQAPGKGLEWVAYISGGGGDTYY |
| | | SSSVKGRFTISRDNAKNSLYLQMNSLRAED |
| | | TAVYYCARHSNPNYYAMDYWGQGTLVTVSS |
| | | ASTKGPSVFPLAPCSRSTSESTAALGCLVK |
| | | DYFPEPVTVSWNSGALTSGVHTFPAVLQSS |
| | | GLYSLSSVVTVPSSSLGTKTYTCNVDHKPS |
| | | NTKVDKRVESKYGPPCPPCPAPEFLGGPSV |
| | | FLFPPKPKDTLMISRTPEVTCVVVDVSQED |
| | | PEVQFNWYVDGVEVHNAKTKPREEQFNSTY |
| | | RVVSVLTVLHQDWLNGKEYKCKVSNKGLPS |
| | | SIEKTISKAKGQPREPQVYTLPPSQEEMTK |
| | | NQVSLTCLVKGFYPSDIAVEWESNGQPENN |
| | | YKTTPPVLDSDGSFFLYSRLTVDKSRWQEG |
| | | NVFSCSVMHEALHNHYTQKSLSLSLG |
| 4 | LC of | EIVLTQSPATLSLSPGERATMSCRASENID |
| | PD1-2 | TSGISFMNWYQQKPGQAPKLLIYVASNQGS |
| | | GIPARFSGSGSGTDFTLTISRLEPEDFAVY |
| | | YCQQSKEVPWTFGQGTKLEIKRTVAAPSVF |
| | | IFPPSDEQLKSGTASVVCLLNNFYPREAKV |
| | | QWKVDNALQSGNSQESVTEQDSKDSTYSLS |
| | | STLTLSKADYEKHKVYACEVTHQGLSSPVT |
| | | KSFNRGEC |
| 5 | HC of | EVMLVESGGGLVQPGGSLRLSCTASGFTFS |
| | PD1-3 | KSAMSWVRQAPGKGLEWVAYISGGGGDTYY |
| | | SSSVKGRFTISRDNAKNSLYLQMNSLRAED |
| | | TAVYYCARHSNVNYYAMDYWGQGTLVTVSS |
| | | ASTKGPSVFPLAPCSRSTSESTAALGCLVK |
| | | DYFPEPVTVSWNSGALTSGVHTFPAVLQSS |
| | | GLYSLSSVVTVPSSSLGTKTYTCNVDHKPS |
| | | NTKVDKRVESKYGPPCPPCPAPEFLGGPSV |
| | | FLFPPKPKDTLMISRTPEVTCVVVDVSQED |
| | | PEVQFNWYVDGVEVHNAKTKPREEQFNSTY |
| | | RVVSVLTVLHQDWLNGKEYKCKVSNKGLPS |
| | | SIEKTISKAKGQPREPQVYTLPPSQEEMTK |
| | | NQVSLTCLVKGFYPSDIAVEWESNGQPENN |
| | | YKTTPPVLDSDGSFFLYSRLTVDKSRWQEG |
| | | NVFSCSVMHEALHNHYTQKSLSLSLG |
| 6 | LC of | EIVLTQSPATLSLSPGERATMSCRASENID |
| | PD1-3 | VSGISFMNWYQQKPGQAPKLLIYVASNQGS |
| | | GIPARFSGSGSGTDFTLTISRLEPEDFAVY |
| | | YCQQSKEVPWTFGQGTKLEIKRTVAAPSVF |
| | | IFPPSDEQLKSGTASVVCLLNNFYPREAKV |
| | | QWKVDNALQSGNSQESVTEQDSKDSTYSLS |
| | | STLTLSKADYEKHKVYACEVTHQGLSSPVT |
| | | KSFNRGEC |
| 7 | HC of | EVMLVESGGGLVQPGGSLRLSCTASGFTFS |
| | PD1-4 | KSAMSWVRQAPGKGLEWVAYISGGGGDTYY |
| | | SSSVKGRFTISRDNAKNSLYLQMNSLRAED |
| | | TAVYYCARHSNVNYYAMDYWGQGTLVTVSS |
| | | ASTKGPSVFPLAPCSRSTSESTAALGCLVK |
| | | DYFPEPVTVSWNSGALTSGVHTFPAVLQSS |
| | | GLYSLSSVVTVPSSSLGTKTYTCNVDHKPS |
| | | NTKVDKRVESKYGPPCPPCPAPEFLGGPSV |
| | | FLFPPKPKDTLMISRTPEVTCVVVDVSQED |
| | | PEVQFNWYVDGVEVHNAKTKPREEQFNSTY |
| | | RVVSVLTVLHQDWLNGKEYKCKVSNKGLPS |
| | | SIEKTISKAKGQPREPQVYTLPPSQEEMTK |
| | | NQVSLTCLVKGFYPSDIAVEWESNGQPENN |
| | | YKTTPPVLDSDGSFFLYSRLTVDKSRWQEG |
| | | NVFSCSVMHEALHNHYTQKSLSLSLG |
| 8 | LC of | EIVLTQSPATLSLSPGERATMSCRASENID |
| | PD1-4 | VSGISFMNWYQQKPGQAPKLLIYVASNQGS |
| | | GIPARFSGSGSGTDFTLTISRLEPEDFAVY |
| | | YCQQSKEVPWTFGQGTKLEIKRTVAAPSVF |
| | | IFPPSDEQLKSGTASVVCLLNNFYPREAKV |
| | | QWKVDNALQSGNSQESVTEQDSKDSTYSLS |
| | | STLTLSKADYEKHKVYACEVTHQGLSSPVT |
| | | KSFNRGEC |
| 9 | HC of | EVMLVESGGGLVQPGGSLRLSCTASGFTFS |
| | PD1-5 | KSAMSWVRQAPGKGLEWVAYISGGGGDTYY |
| | | SSSVKGRFTISRDNAKNSLYLQMNSLRAED |
| | | TAVYYCARHSNVNYYAMDYWGQGTLVTVSS |
| | | ASTKGPSVFPLAPCSRSTSESTAALGCLVK |
| | | DYFPEPVTVSWNSGALTSGVHTFPAVLQSS |
| | | GLYSLSSVVTVPSSSLGTKTYTCNVDHKPS |
| | | NTKVDKRVESKYGPPCPPCPAPEFLGGPSV |
| | | FLFPPKPKDTLMISRTPEVTCVVVDVSQED |
| | | PEVQFNWYVDGVEVHNAKTKPREEQFNSTY |
| | | RVVSVLTVLHQDWLNGKEYKCKVSNKGLPS |
| | | SIEKTISKAKGQPREPQVYTLPPSQEEMTK |
| | | NQVSLTCLVKGFYPSDIAVEWESNGQPENN |
| | | YKTTPPVLDSDGSFFLYSRLTVDKSRWQEG |
| | | NVFSCSVMHEALHNHYTQKSLSLSLG |
| 10 | LC of | EIVLTQSPATLSLSPGERATMSCRASENID |
| | PD1-5 | VSGISFMNWYQQKPGQAPKLLIYVASNQGS |
| | | GIPARFSGSGSGTDFTLTISRLEPEDFAVY |
| | | YCQQSKEVPWTFGQGTKLEIKRTVAAPSVF |
| | | IFPPSDEQLKSGTASVVCLLNNFYPREAKV |
| | | QWKVDNALQSGNSQESVTEQDSKDSTYSLS |
| | | STLTLSKADYEKHKVYACEVTHQGLSSPVT |
| | | KSFNRGEC |

Specifically, the anti-PD-1 antibody molecule described herein above has:

(PD1-1:) a heavy chain comprising the amino acid sequence of SEQ ID NO:1 and a light chain comprising the amino acid sequence of SEQ ID NO:2; or

(PD1-2:) a heavy chain comprising the amino acid sequence of SEQ ID NO:3 and a light chain comprising the amino acid sequence of SEQ ID NO:4; or

(PD1-3:) a heavy chain comprising the amino acid sequence of SEQ ID NO:5 and a light chain comprising the amino acid sequence of SEQ ID NO:6; or

(PD1-4:) a heavy chain comprising the amino acid sequence of SEQ ID NO:7 and a light chain comprising the amino acid sequence of SEQ ID NO:8; or

(PD1-5:) a heavy chain comprising the amino acid sequence of SEQ ID NO:9 and a light chain comprising the amino acid sequence of SEQ ID NO:10.

Atezolizumab (Tecentriq, also known as MPDL3280A) is a phage-derived human IgG1k monoclonal antibody targeting PD-L1 and is described e.g. in Deng et al. mAbs 2016; 8:593-603. It has been approved by the FDA for the treatment of patients suffering from urothelial carcinoma.

Avelumab is a fully human anti-PD-L1 IgG1 monoclonal antibody and described in e.g. Boyerinas et al. Cancer Immunol. Res. 2015; 3:1148-1157.

Durvalumab (MED14736) is a human IgG1k monoclonal antibody with high specificity to PD-L1 and described in e.g. Stewart et al. Cancer Immunol. Res. 2015; 3:1052-1062 or in Ibrahim et al. Semin. Oncol. 2015; 42:474-483.

Further PD-1 antagonists disclosed by Li et al. (supra), or known to be in clinical trials, such as AMP-224, MEDI0680 (AMP-514), REGN2810, BMS-936559, JS001-PD-1, SHR-1210, BMS-936559, TSR-042, JNJ-63723283, MEDI4736, MPDL3280A, and MSB0010718C, may be used as alternative or in addition to the above mentioned antagonists.

The INNs as used herein are also meant to encompass all biosimilar antibodies having the same, or substantially the same, amino acid sequences as the originator antibody, including but not limited to those biosimilar antibodies authorized under 42 USC § 262 subsection (k) in the US and equivalent regulations in other jurisdictions.

All PD-1 antagonists listed above are known in the art with their respective manufacture, therapeutic use and properties. All patent applications referred to above are incorporated by reference in their entirety.

In one embodiment the PD-1 antagonist is pembrolizumab (B1).

In another embodiment the PD-1 antagonist is nivolumab (B2).

In another embodiment the PD-1 antagonist is pidilizumab (B3).

In another embodiment the PD-1 antagonist is atezolizumab (B4).

In another embodiment the PD-1 antagonist is avelumab (B5).

In another embodiment the PD-1 antagonist is durvalumab (B6).

In another embodiment the PD-1 antagonist is PDR-001 (B7).

In another embodiment the PD-1 antagonist is BAP049-Clone-B as defined in table 1 in WO 2015/112900 (page 171) (B8).

In another embodiment the PD-1 antagonist is BAP049-Clone-E as defined in table 1 in WO 2015/112900 (page 171) (B9).

In another embodiment the PD-1 antagonist is selected from the group consisting of BAP058-Clone-K to BAP058-Clone-O as defined in table 1 in WO 2016/061142 (page 265) (B10).

In another embodiment the PD-1 antagonist is PD1-1 (B11).

In another embodiment the PD-1 antagonist is PD1-2 (B12).

In another embodiment the PD-1 antagonist is PD1-3 (B13).

In another embodiment the PD-1 antagonist is PD1-4 (B14).

In another embodiment the PD-1 antagonist is PD1-5 (B15).

All embodiments (B1) to (B15) are preferred embodiments of embodiment (B0) in respect of the nature of the PD-1 antagonist.

To be used in therapy, the respective anti-PD1 and/or anti-PD-L1 antibody molecule, is included into pharmaceutical compositions appropriate to facilitate administration to animals or humans.

For pharmaceutical use, the antibody molecules of the invention may be formulated as a pharmaceutical preparation comprising (i) at least one antibody of the invention and (ii) at least one pharmaceutically acceptable carrier, diluent, excipient, adjuvant, and/or stabilizer, and (iii) optionally one or more further pharmacologically active polypeptides and/or compounds. By “pharmaceutically acceptable” is meant that the respective material does not show any biological or otherwise undesirable effects when administered to an individual and does not interact in a deleterious manner with any of the other components of the pharmaceutical composition (such as e.g. the pharmaceutically active ingredient) in which it is contained. Specific examples can be found in standard handbooks, such as e.g. Remington's Pharmaceutical Sciences, 18^{th} Ed., Mack Publishing Company, USA (1990). For example, the antibodies of the invention may be formulated and administered in any manner known per se for conventional antibodies and antibody fragments and other pharmaceutically active proteins. Thus, according to a further embodiment, the invention relates to a pharmaceutical composition or preparation that contains at least one antibodys of the invention and at least one pharmaceutically acceptable carrier, diluent, excipient, adjuvant and/or stabilizer, and optionally one or more further pharmacologically active substances.

Pharmaceutical preparations for parenteral administration, such as intravenous, intramuscular, subcutaneous injection or intravenous infusion may for example be sterile solutions, suspensions, dispersions, emulsions, or powders which comprise the active ingredient and which are suitable, optionally after a further dissolution or dilution step, for infusion or injection. Suitable carriers or diluents for such preparations for example include, without limitation, sterile water and pharmaceutically acceptable aqueous buffers and solutions such as physiological phosphate-buffered saline, Ringer's solutions, dextrose solution, and Hank's solution; water oils; glycerol; ethanol; glycols such as propylene glycol, as well as mineral oils, animal oils and vegetable oils, for example peanut oil, soybean oil, as well as suitable mixtures thereof.

Solutions of the antibody molecules of the invention may also contain a preservative to prevent the growth of microorganisms, such as antibacterial and antifungal agents, for example, p-hydroxybenzoates, parabens, chlorobutanol, phenol, sorbic acid, thiomersal, (alkali metal salts of) ethylenediamine tetraacetic acid, and the like. In many cases, it will be preferable to include isotonic agents, for example, sugars, buffers or sodium chloride. Optionally, emulsifiers and/or dispersants may be used. The proper fluidity can be maintained, for example, by the formation of liposomes, by the maintenance of the required particle size in the case of dispersions or by the use of surfactants. Other agents delaying absorption, for example, aluminum monostearate and gelatin, may also be added. The solutions may be filled into injection vials, ampoules, infusion bottles, and the like.

In all cases, the ultimate dosage form must be sterile, fluid and stable under the conditions of manufacture and storage. Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Usually, aqueous solutions or suspensions will be preferred. Generally, suitable formulations for therapeutic proteins such as the antibodies of the invention are buffered protein solutions, such as solutions including the protein in a suitable concentration (such as from 0.001 to 400 mg/mL, preferably from 0.005 to 200 mg/mL, more preferably 0.01 to 200 mg/mL, more preferably 1.0-100 mg/mL, such as 1.0 mg/mL (i.v. administration) or 100 mg/mL (s.c. administration) and an aqueous buffer such as:
phosphate buffered saline, pH 7.4,
other phosphate buffers, pH 6.2 to 8.2,
acetate buffers, pH 3.2 to 7.5, preferably pH 4.8 to 5.5
histidine buffers, pH 5.5 to 7.0,
succinate buffers, pH 3.2 to 6.6, and
citrate buffers, pH 2.1 to 6.2,

and, optionally, salts (e.g. NaCl) and/or sugars (such as e.g. sucrose and trehalose) and/or other polyalcohols (such as e.g. mannitol and glycerol) for providing isotonicity of the solution.

Preferred buffered protein solutions are solutions including about 0.05 mg/mL of the antibody of the invention dissolved in 25 mM phosphate buffer, pH 6.5, adjusted to isotonicity by adding 220 mM trehalose. In addition, other agents such as a detergent, e.g. 0.02% Tween-20 or Tween-80, may be included in such solutions. Formulations for subcutaneous application may include significantly higher concentrations of the antibody of the invention, such as up to 100 mg/mL or even above 100 mg/mL. However, it will be clear to the person skilled in the art that the ingredients and the amounts thereof as given above do only represent one, preferred option. Alternatives and variations thereof will be immediately apparent to the skilled person, or can easily be conceived starting from the above disclosure.

The antibody may be administered to the patient at a dose between 1 mg/kg to 20 mg/kg, by one or more separate administrations, or by continuous infusion, e.g. infusion over 1 hour. A typical treatment schedule usually involves administration of the antibody once every week to once every three weeks.

In one embodiment BAP049-Clone-E as defined in table 1 in WO 2015/112900 (page 171) is dosed and administered according to the schedules disclosed in WO 2017/019896 (page 336, last paragraph)

For a more detailed description for PD-1 antagonists already marketed and their use it is referred to the respective Summary of Product Characteristics (incorporated by reference in their entirety).

Combination Therapy

Within this invention it is to be understood that the combinations, compositions, kits, methods, uses or compounds for use according to this invention may envisage the simultaneous, concurrent, sequential, successive, alternate or separate administration of the active ingredients or components. It will be appreciated that the SMAC mimetic and the PD-1 antagonist can be administered formulated either dependently or independently, such as e.g. the SMAC mimetic and the PD-1 antagonist may be administered either as part of the same pharmaceutical composition/dosage form or, preferably, in separate pharmaceutical compositions/dosage forms.

In this context, “combination” or “combined” within the meaning of this invention includes, without being limited, a product that results from the mixing or combining of more than one active ingredient and includes both fixed and non-fixed (e.g. free) combinations (including kits) and uses, such as e.g. the simultaneous, concurrent, sequential, successive, alternate or separate use of the components or ingredients. The term “fixed combination” means that the active ingredients are both administered to a patient simultaneously in the form of a single entity or dosage. The term “non-fixed combination” means that the active ingredients are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active ingredients.

The administration of the SMAC mimetic and PD-1 antagonist may take place by co-administering the active components or ingredients, such as e.g. by administering them simultaneously or concurrently in one single or in two separate formulations or dosage forms. Alternatively, the administration of the SMAC mimetic and the PD-1 antagonist may take place by administering the active components or ingredients sequentially or in alternation, such as e.g. in two separate formulations or dosage forms.

For example, simultaneous administration includes administration at substantially the same time. This form of administration may also be referred to as “concomitant” administration. Concurrent administration includes administering the active agents within the same general time period, for example on the same day(s) but not necessarily at the same time. Alternate administration includes administration of one agent during a time period, for example over the course of a few days or a week, followed by administration of the other agent during a subsequent period of time, for example over the course of a few days or a week, and then repeating the pattern for one or more cycles. Sequential or successive administration includes administration of one agent during a first time period (for example over the course of a few days or a week) using one or more doses, followed by administration of the other agent during a second time period (for example over the course of a few days or a week) using one or more doses. An overlapping schedule may also be employed, which includes administration of the active agents on different days over the treatment period, not necessarily according to a regular sequence. Variations on these general guidelines may also be employed, e.g. according to the agents used and the condition of the subject.

The aforementioned applies accordingly if the combination setting is not a dual but a triple or higher multiple combination approach.

The elements of the combinations of this invention may be administered (whether dependently or independently) by methods customary to the skilled person, e.g. by oral, enterical, parenteral (e.g., intramuscular, intraperitoneal, intravenous, transdermal or subcutaneous injection, or implant), nasal, vaginal, rectal, or topical routes of administration and may be formulated, alone or together, in suitable dosage unit formulations containing conventional non-toxic pharmaceutically acceptable carriers, excipients and/or vehicles appropriate for each route of administration.

Accordingly, in one aspect of the invention, the invention provides a method of treating and/or preventing an oncological or hyperproliferative disease, in particular cancer, as described herein, comprising administering to a patient in need thereof a therapeutically effective amount of a SMAC mimetic and a therapeutically effective amount of a PD-1 antagonist and, optionally, one or more additional therapeutic agent(s), each as described herein, wherein the SMAC mimetic is administered simultaneously, concurrently, sequentially, successively, alternately or separately with the PD-1 antagonist and the optional one or more additional therapeutic agent(s) if present.

In another aspect, the invention provides a SMAC mimetic as described herein for use in a method of treating and/or preventing an oncological or hyperproliferative disease, in particular cancer, as described herein, said method comprising administering the SMAC mimetic in combination with a PD-1 antagonist and, optionally, one or more additional therapeutic agent(s), each as described herein, wherein the SMAC mimetic is administered simultaneously, concurrently, sequentially, successively, alternately or separately with the PD-1 antagonist and the optional one or more additional therapeutic agent(s) if present.

In another aspect, the invention provides a PD-1 antagonist as described herein for use in a method of treating and/or preventing an oncological or hyperproliferative disease, in particular cancer, as described herein, said method comprising administering the PD-1 antagonist in combination with a SMAC mimetic and, optionally, one or more additional therapeutic agent(s), each as described herein, wherein the PD-1 antagonist is administered simultaneously, concurrently, sequentially, successively, alternately or separately with the SMAC mimetic and the optional one or more additional therapeutic agent(s) if present.

In another aspect, the invention provides the use of a SMAC mimetic as described herein for preparing a pharmaceutical composition for use in a method of treating and/or preventing an oncological or hyperproliferative disease, in particular cancer, as described herein, wherein the SMAC mimetic is to be used in combination with a PD-1 antagonist and, optionally, one or more additional therapeutic agent(s), each as described herein, and wherein the SMAC mimetic is to be administered simultaneously, concurrently, sequentially, successively, alternately or separately with the PD-1 antagonist and the optional one or more additional therapeutic agent(s) if present.

In another aspect, the invention provides the use of PD-1 antagonist as described herein for preparing a pharmaceutical composition for use in a method of treating and/or preventing an oncological or hyperproliferative disease, in particular cancer, as described herein, wherein the PD-1 antagonist is to be used in combination with a SMAC mimetic and, optionally, one or more additional therapeutic agent(s), each as described herein, and wherein the PD-1 antagonist is to be administered simultaneously, concurrently, sequentially, successively, alternately or separately with the SMAC mimetic and the optional one or more additional therapeutic agent(s) if present.

In another aspect, the invention provides a kit comprising
a first pharmaceutical composition or dosage form comprising a SMAC mimetic as described herein, and, optionally, one or more pharmaceutically acceptable carriers, excipients and/or vehicles,
a second pharmaceutical composition or dosage form comprising a PD-1 antagonist as described herein, and, optionally, one or more pharmaceutically acceptable carriers, excipients and/or vehicles, and, optionally
one or more additional pharmaceutical composition(s) or dosage form(s) each comprising one additional therapeutic agent as described herein, and, optionally, one or more pharmaceutically acceptable carriers, excipients and/or vehicles,

for use in a method of treating and or/preventing an oncological or hyperproliferative disease, in particular cancer, as described herein, wherein the first pharmaceutical composition is to be administered simultaneously, concurrently, sequentially, successively, alternately or separately with the second pharmaceutical composition or dosage form and the optional one or more additional pharmaceutical composition(s) or dosage form(s) if present.

In a further embodiment of the invention, the components (i.e. the combination partners) of the combinations, kits, uses, methods and compounds for use according to the invention (including all embodiments) are administered simultaneously.

In a further embodiment of the invention, the components (i.e. the combination partners) of the combinations, kits, uses, methods and compounds for use according to the invention (including all embodiments) are administered concurrently.

In a further embodiment of the invention, the components (i.e. the combination partners) of the combinations, kits, uses, methods and compounds for use according to the invention (including all embodiments) are administered sequentially.

In a further embodiment of the invention, the components (i.e. the combination partners) of the combinations, kits, uses, methods and compounds for use according to the invention (including all embodiments) are administered successively.

In a further embodiment of the invention, the components (i.e. the combination partners) of the combinations, kits, uses, methods and compounds for use according to the invention (including all embodiments) are administered alternately.

In a further embodiment of the invention, the components (i.e. the combination partners) of the combinations, kits, uses, methods and compounds for use according to the invention (including all embodiments) are administered separately.

In a preferred embodiment, the SMAC mimetic as described herein is to be administered orally.

In another preferred embodiment, the PD-1 antagonist as described herein is to be administered intravenously.

The “therapeutically effective amount” of the active compound(s) to be administered is the minimum amount necessary to prevent, ameliorate, or treat a disease or disorder.

The combinations of this invention may be administered at therapeutically effective single or divided daily doses. The active components of the combination may be administered in such doses which are therapeutically effective in monotherapy, or in such doses which are lower than the doses used in monotherapy, but when combined result in a desired (joint) therapeutically effective amount.

The permutation of embodiments (A0) to (A27) (in respect of the SMAC mimetic) with embodiments (B0) to (B15) (in respect of the PD-1 antagonist) results in 448 specific dual combinations C0 to C447 (C0=A0B0, C1=A0B1, C2=A0B2, . . . etc.) which shall all be deemed to be specifically disclosed and to be embodiments of the invention and of all of its combinations, compositions, kits, methods, uses and compounds for use.

Additional Therapeutic Agent(s)

The combinations, compositions, kits, uses, methods and compounds for use according to the present invention (including all embodiments) including a SMAC mimetic and a PD-1 antagonist, both as described herein, may optionally include one or more additional therapeutic agent(s).

This/these additional therapeutic agent(s) may (each) be selected from the following (without being limited thereto):
an immunotherapeutic agent, such as modulators of the following checkpoint inhibitors: TIM3, PD-L1, PD-L2, CTLA-4, VISTA, BTLA, TIGIT, CD160, LAIR1, 2B4, CEACAM;
a cancer vaccine;
a DNA damaging agent;
an inhibitor of angiogenesis;
an inhibitor of signal transduction pathways;
an inhibitor of mitotic checkpoints; and

hormones, hormone analogues and antihormones (e.g. tamoxifen, toremifene, raloxifene, fulvestrant, megestrol acetate, flutamide, nilutamide, bicalutamide, aminoglutethimide, cyproterone acetate, finasteride, buserelin acetate, fludrocortisone, fluoxymesterone, medroxyprogesterone, octreotide), aromatase inhibitors (e.g. anastrozole, letrozole, liarozole, vorozole, exemestane, atamestane), LHRH agonists and antagonists (e.g. goserelin acetate, luprolide), inhibitors of growth factors (growth factors such as for example “platelet derived growth factor (PDGF)”, “fibroblast growth factor (FGF)”, “vascular endothelial growth factor (VEGF)”, “epidermal growth factor (EGF)”, “insuline-like growth factors (IGF)”, “human epidermal growth factor (HER, e.g. HER2, HER3, HER4)” and “hepatocyte growth factor (HGF)”), inhibitors are for example “growth factor” antibodies, “growth factor receptor” antibodies and tyrosine kinase inhibitors, such as for example cetuximab, gefitinib, imatinib, lapatinib, bosutinib and trastuzumab); antimetabolites (e.g. antifolates such as methotrexate, raltitrexed, pyrimidine analogues such as 5-fluorouracil (5-FU), capecitabine and gemcitabine, purine and adenosine analogues such as mercaptopurine, thioguanine, cladribine and pentostatin, cytarabine (ara C), fludarabine); antitumour antibiotics (e.g. anthracyclins such as doxorubicin, doxil (pegylated liposomal doxorubicin hydrochloride, myocet (non-pegylated liposomal doxorubicin), daunorubicin, epirubicin and idarubicin, mitomycin-C, bleomycin, dactinomycin, plicamycin, streptozocin); platinum derivatives (e.g. cisplatin, oxaliplatin, carboplatin); alkylation agents (e.g. estramustin, meclorethamine, melphalan, chlorambucil, busulphan, dacarbazin, cyclophosphamide, ifosfamide, temozolomide, nitrosoureas such as for example carmustin and lomustin, thiotepa); antimitotic agents (e.g. Vinca alkaloids such as for example vinblastine, vindesin, vinorelbin and vincristine; and taxanes such as paclitaxel, docetaxel); angiogenesis inhibitors (e.g. tasquinimod), tubuline inhibitors; DNA synthesis inhibitors (e.g. sapacitabine), PARP inhibitors, topoisomerase inhibitors (e.g. epipodophyllotoxins such as for example etoposide and etopophos, teniposide, amsacrin, topotecan, irinotecan, mitoxantrone), serine/threonine kinase inhibitors (e.g. PDK 1 inhibitors, Raf inhibitors, A-Raf inhibitros, B-Raf inhibitors, C-Raf inhibitors, mTOR inhibitors, mTORC1/2 inhibitors, P13K inhibitors, PI3Kα inhibitors, dual mTOR/P13K inhibitors, STK 33 inhibitors, AKT inhibitors, PLK 1 inhibitors, inhibitors of CDKs, Aurora kinase inhibitors), tyrosine kinase inhibitors (e.g. PTK2/FAK inhibitors), protein protein interaction inhibitors (e.g. IAP activator, Mcl-1, MDM2/MDMX), MEK inhibitors (e.g. pimasertib), ERK inhibitors, FLT3 inhibitors (e.g. quizartinib), BRD4 inhibitors, IGF-1R inhibitors, TRAILR2 agonists, Bcl-xL inhibitors, Bcl-2 inhibitors (e.g. venetoclax), Bcl-2/Bcl-xL inhibitors, ErbB receptor inhibitors, BCR-ABL inhibitors, ABL inhibitors, Src inhibitors, rapamycin analogs (e.g. everolimus, temsirolimus, ridaforolimus, sirolimus), androgen synthesis inhibitors (e.g. abiraterone, TAK-700), androgen receptor inhibitors (e.g. enzalutamide, ARN-509), immunotherapy (e.g. sipuleucel-T), DNMT inhibitors (e.g. SGI 110, temozolomide, vosaroxin), HDAC inhibitors (e.g. vorinostat, entinostat, pracinostat, panobinostat), ANG1/2 inhibitors (e.g. trebananib), CYP17 inhibitors (e.g. galeterone), radiopharmaceuticals (e.g. radium-223, alpharadin), immunotherapeutic agents (e.g. poxvirus-based vaccine, ipilimumab, immune checkpoint inhibitors) and various chemotherapeutic agents such as amifostin, anagrelid, clodronat, filgrastin, interferon, interferon alpha, leucovorin, rituximab, procarbazine, levamisole, mesna, mitotane, pamidronate and porfimer;

2-chlorodesoxyadenosine, 2-fluorodesoxycytidine, 2-methoxyoestradiol, 2C4, 3-alethine, 131-I-TM-601, 3CPA, 7-ethyl-10-hydroxycamptothecin, 16-aza-epothilone B, ABT-199, ABT-263/navitoclax, ABT-737, A 105972, A 204197, aldesleukin, alisertib/MLN8237, alitretinoin, allovectin-7, altretamine, alvocidib, amonafide, anthrapyrazole, AG-2037, AP-5280, apaziquone, apomine, aranose, arglabin, arzoxifene, atamestane, atrasentan, auristatin PE, AVLB, AZ10992, ABX-EGF, AMG-479 (ganitumab), AMG-232, AMG-511, AMG 2520765, AMG 2112819, ARRY 162, ARRY 438162, ARRY-300, ARRY-142886/AZD-6244 (selumetinib), ARRY-704/AZD-8330, ATSP-7041, AR-12, AR-42, AS-703988, AXL-1717, AZD-1480, AZD-4547, AZD-8055, AZD-5363, AZD-6244, AZD-7762, ARQ-736, ARQ 680, AS-703026 (primasertib), avastin, AZD-2014, azacitidine (5-aza), azaepothilone B, azonafide, barasertib/AZD1152, BAY-43-9006, BAY 80-6946, BBR-3464, BBR-3576, bevacizumab, BEZ-235/dactolisib, biricodar dicitrate, birinapant, BCX-1777, BKM-120/buparlisib, bleocin, BLP-25, BMS-184476, BMS-247550, BMS-188797, BMS-275291, BMS-663513, BMS-754807, BNP-1350, BNP-7787, BIBW 2992/afatinib, BIBF 1120/nintedanib, BI 836845, BI 2536, BI 6727/volasertib, BI 836845, BI 847325, BI 853520, BIIB-022, bleomycinic acid, bleomycin A, bleomycin B, brivanib, bryostatin-1, bortezomib, brostallicin, busulphan, BYL-719/alpelisib, CA-4 prodrug, CA-4, cabazitaxel, cabozantinib, CapCell, calcitriol, canertinib, canfosfamide, capecitabine, carboxyphthalatoplatin, CCI-779, CC-115, CC-223, CEP-701, CEP-751, CBT-1 cefixime, ceflatonin, ceftriaxone, celecoxib, celmoleukin, cemadotin, CGM-097, CH4987655/RO-4987655, chlorotrianisene, cilengitide, ciclosporin, CD20 antibodies, CDA-II, CDC-394, CKD-602, CKI-27, clofarabine, colchicin, combretastatin A4, COT inhibitors, CHS-828, CH-5132799, CLL-Thera, CMT-3 cryptophycin 52, CPI-613, CTP-37, CTLA-4 monoclonal antibodies (e.g. ipilimumab), CP-461, crizotinib, CV-247, cyanomorpholinodoxorubicin, cytarabine, D 24851, dasatinib, decitabine, deoxorubicin, deoxyrubicin, deoxycoformycin, depsipeptide, desoxyepothilone B, dexamethasone, dexrazoxanet, diethylstilbestrol, diflomotecan, didox, DMDC, dolastatin 10, doranidazole, DS-7423, DS-3032, E7010, E-6201, edatrexat, edotreotide, efaproxiral, eflornithine, EGFR inhibitors, EKB-569, EKB-509, enzastaurin, elesclomol, elsamitrucin, epothilone B, epratuzumab, EPZ-004777, ER-86526, erlotinib, ET-18-OCH3, ethynylcytidine, ethynyloestradiol, exatecan, exatecan mesylate, exemestane, exisulind, fenretinide, figitumumab, floxuridine, folic acid, FOLFOX, FOLFOX4, FOLFIRI, formestane, fostamatinib, fotemustine, galarubicin, gallium maltolate, ganetespib, gefinitib, gemtuzumab, gemtuzumab ozogamicin, gimatecan, glufosfamide, GCS-IOO, GDC-0623, GDC-0941 (pictrelisib), GDC-0980, GDC-0032, GDC-0068, GDC-0349, GDC-0879, G17DT immunogen, GMK, GMX-1778, GPX-100, gp100-peptide vaccines, GSK-5126766, GSK-690693, GSK-1120212 (trametinib), GSK-1995010, GSK-2118436 (dabrafenib), GSK-2126458, GSK-2132231A, GSK-2334470, GSK-2110183, GSK-2141795, GSK-2636771, GSK-525762A/I-BET-762, GW2016, granisetron, herceptine, hexamethylmelamine, histamine, homoharringtonine, hyaluronic acid, hydroxyurea, hydroxyprogesterone caproate, HDM-201, ibandronate, ibritumomab, ibrutinib/PCI-32765, idasanutlin, idatrexate, idelalisib/CAL-101, idenestrol, IDN-5109, IGF-1R inhibitors, IMC-1C11, IMC-A12 (cixutumumab), immunol, indisulam, interferon alpha-2a, interferon alpha-2b, pegylated interferon alpha-2b, interleukin-2, INK-1117, INK-128, INSM-18, ionafarnib, iproplatin, irofulven, isohomohalichondrin-B, isoflavone, isotretinoin, ixabepilone, JRX-2, JSF-154, JQ-1, J-107088, conjugated oestrogens, kahalid F, ketoconazole, KW-2170, KW-2450, KU-55933, LCL-161, lobaplatin, leflunomide, lenalidomide, lenograstim, leuprolide, leuporelin, lexidronam, LGD-1550, linezolid, lovastatin, lutetium texaphyrin, lometrexol, lonidamine, losoxantrone, LU 223651, lurbinectedin, lurtotecan, LY-S6AKT1, LY-2780301, LY-2109761/galunisertib, mafosfamide, marimastat, masoprocol, mechloroethamine, MEK inhibitors, MEK-162, methyltestosteron, methylprednisolone, MEDI-573, MEN-10755, MDX-H210, MDX-447, MDX-1379, MGV, midostaurin, minodronic acid, mitomycin, mivobulin, MK-2206, MK-0646 (dalotuzumab), MLN518, MLN-0128, MLN-2480, motexafin gadolinium, MS-209, MS-275, MX6, neridronate, neratinib, Nexavar, neovastat, nilotinib, nimesulide, nitroglycerin, nolatrexed, norelin, N-acetylcysteine, NU-7441 06-benzylguanine, oblimersen, omeprazole, olaparib, oncophage, oncoVEX^{GM-CSF}, ormiplatin, ortataxel, OX44 antibodies, OSI-027, OSI-906 (linsitinib), 4-1BB antibodies, oxantrazole, oestrogen, onapristone, palbociclib/PD-0332991, panitumumab, panobinostat, patupilone, pazopanib, pegfilgrastim, PCK-3145, pegfilgrastim, PBI-1402, PBI-05204, PD0325901, PD-1 and PD-L1 antibodies (e.g. pembrolizumab, nivolumab, pidilizumab, MEDI-4736/durvalumab, RG-7446/atezolizumab), PD-616, PEG-paclitaxel, albumin-stabilized paclitaxel, PEP-005, PF-05197281, PF-05212384, PF-04691502, PF-3758309, PHA-665752, PHT-427, P-04, PKC412, P54, PI-88, pelitinib, pemetrexed, pentrix, perifosine, perillylalcohol, pertuzumab, pevonedistat, P13K inhibitors, PI3K/mTOR inhibitors, PG-TXL, PG2, PLX-4032/RO-5185426 (vemurafenib), PLX-3603/RO-5212054, PT-100, PWT-33597, PX-866, picoplatin, pivaloyloxymethylbutyrate, pixantrone, phenoxodiol O, PKI166, plevitrexed, plicamycin, polyprenic acid, ponatinib, porfiromycin, posaconazole, prednisone, prednisolone, PRT-062607, quinamed, quinupristin, quizartinib/AC220, R115777, RAF-265, ramosetron, ranpirnase, RDEA-119/BAY 869766, RDEA-436, rebeccamycin analogues, receptor tyrosine kinase (RTK) inhibitors, revimid, RG-7167, RG-7112, RG-7304, RG-7421, RG-7321, RG-7356, RG 7440, RG-7775, rhizoxin, rhu-MAb, rigosertib rinfabate, risedronate, rituximab, robatumumab, rofecoxib, romidepsin, RO-4929097, RO-31-7453, RO-5126766, RO-5068760, RPR 109881A, rubidazone, rubitecan, R-flurbiprofen, RX-0201, ruxolitinib, S-9788, sabarubicin, SAHA, sapacitabine, SAR-405838, sargramostim, satraplatin, SB-408075, SB-431542, Se-015Ne-015, SU5416, SU6668, SDX-101, selinexor, semustin, seocalcitol, SM-11355, SN-38, SN-4071, SR-27897, SR-31747, SR-13668, SRL-172, sorafenib, spiroplatin, squalamine, STF-31, suberanilohydroxamic acid, sutent, T 900607, T 138067, TAE-684, TAK-733, TAS-103, tacedinaline, talaporfin, tanespimycin, Tarceva, tariquitar, tasisulam, taxotere, taxoprexin, tazarotene, tegafur, temozolamide, tesmilifene, testosterone, testosterone propionate, tesmilifene, tetraplatin, tetrodotoxin, tezacitabine, thalidomide, theralux, therarubicin, thymalfasin, thymectacin, tiazofurin, tipifarnib, tirapazamine, tocladesine, tomudex, toremofin, tosedostat. trabectedin, TransMlD-107, transretinic acid, traszutumab, tremelimumab, tretinoin, triacetyluridine, triapine, triciribine, trimetrexate, TLK-286TXD 258, tykerb/tyverb, urocidin, valproic acid, valrubicin, vandetanib, vatalanib, vincristine, vinflunine, virulizin, vismodegib, vosaroxin, WX-UK1, WX-554, vectibix, XAV-939, xeloda, XELOX, XL-147, XL-228, XL-281, XL-518/R-7420/GDC-0973, XL-765, YM-511, YM-598, ZD-4190, ZD-6474, ZD-4054, ZD-0473, ZD-6126, ZD-9331, ZD1839, ZSTK-474, zoledronat and zosuquidar.

Oncological or Hyperproliferative Diseases/Cancers

The combinations, compositions, kits, uses, methods and compounds for use according to the present invention (including all embodiments) are useful for the treatment and/or prevention of oncological and hyperproliferative disorders.

In certain embodiments the combinations, compositions, kits, uses, methods and compounds for use according to the present invention (including all embodiments) are useful for the treatment of oncological and hyperproliferative disorders.

In certain embodiments, the hyperproliferative disorder is cancer.

Cancers are classified in two ways: by the type of tissue in which the cancer originates (histological type) and by primary site, or the location in the body, where the cancer first developed. The most common sites in which cancer develops include the skin, lung, breast, prostate, colon and rectum, cervix and uterus as well as the hematological compartment.

The combinations, compositions, kits, uses, methods and compounds for use according to the invention (including all embodiments) may be useful in the treatment of a variety of oncological and hyperproliferative disorders, in particular cancers, including, for example, but not limited to the following:
brain related cancer such as adult brain tumour, childhood brain stem glioma, childhood cerebellar astrocytoma, childhood cerebral astrocytoma/malignant glioma, childhood ependymoma, childhood medulloblastoma, childhood supratentorial primitive neuroectodermal tumours, childhood visual pathway and hypothalamic glioma and other childhood brain tumours;
breast cancer;
digestive/gastrointestinal related cancer such as anal cancer, extrahepatic bile duct cancer, gastrointestinal carcinoid tumour, cholangiocarcinoma, colon cancer, esophageal cancer, gallbladder cancer, adult primary liver cancer (hepatocellular carcinoma, hepatoblastoma) childhood liver cancer, pancreatic cancer, rectal cancer, small intestine cancer and stomach (gastric) cancer;
endocrine related cancer such as adrenocortical carcinoma, gastrointestinal carcinoid tumour, islet cell carcinoma (endocrine pancreas), parathyroid cancer, pheochromocytoma, pituitary tumour and thyroid cancer;
eye related cancer such as intraocular melanoma, and retinoblastoma;
genitourinary related cancer such as bladder cancer, kidney (renal cell) cancer, penile cancer, prostate cancer, transitional cell renal pelvis and ureter cancer, testicular cancer, urethral cancer, Wilms' tumour and other childhood kidney tumours;
germ cell related cancer such as childhood extracranial germ cell tumour, extragonadal germ cell tumour, ovarian germ cell tumour and testicular cancer;
gynecologic cancer such as cervical cancer, endometrial cancer, gestational trophoblastic tumour, ovarian epithelial cancer, ovarian germ cell tumour, ovarian low malignant potential tumour, uterine sarcoma, vaginal cancer and vulvar cancer;
head and neck related cancer such as hypopharyngeal cancer, laryngeal cancer, lip and oral cavity cancer, metastatic squamous neck cancer with occult primary, nasopharyngeal cancer, oropharyngeal cancer, paranasal sinus and nasal cavity cancer (e.g. sinonasal squamouns cell carcinoma), parathyroid cancer and salivary gland cancer;
hematologic/blood related cancer such as leukemias, such as adult acute lymphoblastic leukemia, childhood acute lymphoblastic leukemia, adult acute myeloid leukemia, childhood acute myeloid leukemia, chronic lymphocytic leukemia, chronic myelogenous leukemia and hairy cell leukemia; and lymphomas, such as AIDS-related lymphoma, cutaneous T-cell lymphoma, adult Hodgkin's lymphoma, childhood Hodgkin's lymphoma, Hodgkin's lymphoma during pregnancy, mycosis fungoides, adult non-Hodgkin's lymphoma, childhood non-Hodgkin's lymphoma, non-Hodgkin's lymphoma during pregnancy, primary central nervous system lymphoma, Sezary syndrome, cutaneous T-cell lymphoma and Waldenström's macroglobulinemia and other hematologic/blood related cancer such as chronic myeloproliferative disorders, multiple myeloma/plasma cell neoplasm, myelodysplastic syndromes and myelodysplastic/myeloproliferative diseases;
musculoskeletal related cancer such as Ewing's family of tumours, osteosarcoma, malignant fibrous histiocytoma of bone, childhood rhabdomyosarcoma, adult soft tissue sarcoma, childhood soft tissue sarcoma and uterine sarcoma; hemangiosarcomas and angiosarcoma;
neurologic related cancer such as adult brain tumour, childhood brain tumour, brain stem glioma, cerebellar astrocytoma, cerebral astrocytoma/malignant glioma, ependmoma, medulloblastoma, supratentorial primitive neuroectodermal tumours, visual pathway and hypothalamic glioma and other brain tumours such as neuroblastoma, pituitary tumour and primary central nervous system lymphoma;
respiratory/thoracic related cancer such as non-small cell lung cancer (NSCLC), small cell lung cancer (SCLC), squamous cell carcinoma (SCC) of the lung, malignant mesothelioma, thymoma and thymic carcinoma;
skin related cancer such as cutaneous T-cell lymphoma, Kaposi's sarcoma, melanoma, Merkel cell carcinoma and skin cancer;
small blue round cell tumours.

In a further embodiment, the combinations, compositions, kits, uses, methods and compounds for use of the invention (including all embodiments) are beneficial in the treatment of cancers of the hematopoietic system including leukemias, lymphomas and myelomas, cancers of the gastrointestinal tract including esophageal, gastric, colorectal, pancreatic, liver and gall bladder and bile duct cancer; kidney, prostate and bladder cancer; gynecological cancers including breast, ovarian, cervical and endometrial cancer; skin and head and neck cancers including malignant melanomas; pediatric cancers like Wilms' tumour, neuroblastoma and Ewing' sarcoma; brain cancers like glioblastoma; sarcomas like osteosarcoma, soft tissue sarcoma, rhabdomyosarcoma, hemangiosarcoma; lung cancer including non-small cell lung cancer, mesothelioma and thyroid cancer.

In a further embodiment of the invention, the combinations, compositions, kits, uses, methods and compounds for use according to the invention (including all embodiments) are used to treat non-small cell lung cancer (NSCLC) (including for example locally advanced or metastatic NSCLC (stage IIIB/IV), NSCLC adenocarcinoma, NSCLC with squamous histology, NSCLC with non-squamous histology).

In a further embodiment of the invention, the combinations, compositions, kits, uses, methods and compounds for use according to the invention (including all embodiments) are used in the treatment of non-small cell lung cancer (NSCLC), in particular NSCLC adenocarcinoma.

In a further embodiment of the invention, the combinations, compositions, kits, uses, methods and compounds for use according to the invention (including all embodiments) are used in the treatment of multiple myeloma (MM).

In a further embodiment of the invention, the combinations, compositions, kits, uses, methods and compounds for use according to the invention (including all embodiments) are used in the treatment of breast cancer, in particular triple-negative breast cancer (TNBC).

In a further embodiment of the invention, the combinations, compositions, kits, uses, methods and compounds for use according to the invention (including all embodiments) are used in the treatment of cancer patients who are treatment naïve in respect of treatment with a checkpoint inhibitor or immunomodulator, i.e., e.g., patients who are treatment naïve in respect of treatment with a PD-1 antagonist.

In a further embodiment of the invention, the combinations, compositions, kits, uses, methods and compounds for use according to the invention (including all embodiments) are used in the treatment of cancer patients who relapsed during treatment with a checkpoint inhibitor or immunomodulator, i.e., e.g., patients who relapsed during treatment with a PD-1 antagonist.

The therapeutic applicability of the combination therapy according to this invention may include first line, second line, third line or further lines of treatment of patients. The cancer may be metastatic, recurrent, relapsed, resistant or refractory to one or more anti-cancer treatments. Thus, the patients may be treatment naïve, or may have received one or more previous anti-cancer therapies, which have not completely cured the disease.

Patients with relapse and/or with resistance to one or more anti-cancer agents (e.g. the single components of the combination, or standard chemotherapeutics) are also amenable for combined treatment according to this invention, e.g. for second or third line treatment cycles (optionally in further combination with one or more other anti-cancer agents), e.g. as add-on combination or as replacement treatment.

Accordingly, some of the disclosed combination therapies of this invention are effective at treating subjects whose cancer has relapsed, or whose cancer has become drug resistant or multi-drug resistant, or whose cancer has failed one, two or more lines of mono- or combination therapy with one or more anti-cancer agents (e.g. the single components of the combination, or standard chemotherapeutics).

A cancer which initially responded to an anti-cancer drug can relapse and it becomes resistant to the anti-cancer drug when the anti-cancer drug is no longer effective in treating the subject with the cancer, e.g. despite the administration of increased dosages of the anti-cancer drug. Cancers that have developed resistance to two or more anti-cancer drugs are said to be multi-drug resistant.

Accordingly, in some methods of combination treatment of this invention, treatment with a combination according to this invention administered secondly or thirdly is begun if the patient has resistance or develops resistance to one or more agents administered initially or previously. The patient may receive only a single course of treatment with each agent or multiple courses with one, two or more agents.

In certain instances, combination therapy according to this invention may hence include initial or add-on combination, replacement or maintenance treatment.

In a further embodiment of the invention, the combinations, compositions, kits, uses, methods and compounds for use according to the invention (including all embodiments) are used in the treatment of cancers/cancer patients (suffering from cancers as described herein, in particular suffering from NSCLC as described herein) which are treatment naïve, i.e. their cancer disease has not been treated previously. In further embodiments the cancers/cancer patients (suffering from cancers as described herein, in particular suffering from NSCLC as described herein) have been previously treated with one or more immune checkpoint inhibitor and/or immuno modulator, e.g. one or more PD-1 antagonist(s).

The present invention is not to be limited in scope by the specific embodiments described herein. Various modifications of the invention in addition to those described herein may become apparent to those skilled in the art from the present disclosure. Such modifications are intended to fall within the scope of the appended claims.

All patent applications cited herein are hereby incorporated by reference in their entireties.

### REFERENCES

1. Damgaard R, Gyrd-Hansen M. Inhibitor of apoptosis (IAP) proteins in regulation of inflammation and innate immunity, Discovery Medicine; Denmark. 2011; 11 (58): 221-231.
2. DiDonato J, Mercurio F, Karin M. NF-κB and the link between inflammation and cancer, Immunological reviews; Singapore. 2012; 246 (1): 379-400.
3. Li L, Thomas R, Suzuki H, Brabander J, Wang X, Harran P. A Small Molecule Smac Mimic Potentiates TRAIL- and TNFα-Mediated Cell Death, Science; 2004; 305 (5689): 1471-1474.
4. Benetatos C, Mitsuuchi Y, Burns J, Neiman E, Condon S, Yu G, Seipel M, Kapoor G, LaPorte M, Rippin S, Deng Y, Hendi M, Tirunahari P, Lee Y, Haimowitz T, Alexander M, Graham M, Weng D, Shi Y, McKinlay M, Chunduru S. Birinapant (TL32711), a Bivalent SMAC Mimetic, Targets TRAF2-Associated cIAPs, Abrogates TNF-Induced NF-κB Activation, and Is Active in Patient-Derived Xenograft Models, Molecular Cancer Therapeutics; 2014; 13 (4): 867-879.
5. Cheung H, Mahiney D, LaCasse E, Korneluk R. Down-regulation of c-FLIP Enhances Death of Cancer Cells by Smac Mimetic Compound, Cancer Research; Ottawa. 2009; 69 (19): 7729-7738.
6. Aaes T, Kaczmarek A, Delvaeye T, De Crane B, De Koker S, Heyndrickx L, Delrue I, Taminau J, Wiernicki B, De Groote P, Garg A, Leybaert L, Grooten J, Bertrand M, Agostinis P, Berx G, Declercq W, Vandenabeele P, Krysko D. Vaccination with Necroptotic Cancer Cellslnduces Efficient Anti-tumor Immunity, Cell Report; Ghent, Leuven. 2016; 15 (2): 274-287.
7. Beug S, Conrad S, Alain T, Korneluk R, LaCasse E. Combinatorial cancer immunotherapy strategies with proapoptotic small-molecule IAP antagonists, International Journal of Developmental Biology; Ottawa. 2015; 59: 141-147.
8. Infante J, Dees E, Olszanski A, Dhuria S, Sen S, Cameron S, Cohen R. Phase I Dose-Escalation Study of LCL161, an Oral Inhibitor of Apoptosis Proteins Inhibitor, in Patients With Advanced Solid Tumors, Journal of Clinical Oncology; 2014; 32 (28): 3103-3110.

### Methods

### Example 1

Anti-Tumor Activity of the Exemplary SMAC Mimetic BIA-1 in Combination with RMP1-14, a Mouse Tool Antibody to PD-1, in a Subcutaneous Syngeneic Mouse Model Derived from the Breast Cancer Cell Line EMT6 in Balb/c Mice

The efficacy of the exemplary SMAC mimetic BIA-1 was tested in a s.c. cell line derived syngeneic model of mouse breast cancer (EMT6) as single agent and in combination with RMP1-14, a mouse antibody to PD-1 (BioXCell # BE0146).

BALB/cJBomTac mice were used in this study. 1×10⁶ EMT6 breast cancer cells were injected per mouse to establish a tumor. Tumor volume was measured at least three times per week using a caliper. Treatment started when tumors had reached a median tumor volume of 71-231 mm³ and was terminated after 33 days.

Ten tumor-bearing animals were treated with the exemplary SMAC mimetic BIA-1 per os (p.o.) daily and twice weekly i.p. with RMP1-14 or a combination of both compounds. Ten animals were used in the vehicle/isotype control-treated group. Animals were euthanized at the end of the study for ethical reasons based on the tumor mass (tumor ≥1.5 cm³).

Cells

EMT6 cells were obtained from ATCC (catalog number ATCC® CRL2755™). A master cell bank (MCB) and a working cell bank (WCB) were established. Cells were cultured in T175 tissue culture flasks at 37° C. and 5% CO₂. The medium used was Waymouth's MB 752/1 supplemented with 15% fetal calf serum (HyClone® Fetal Bovine Serum Characterized; Cat No SH30071.03; by Thermo Scientific), and 2 mM L-Glutamine (L-Glutamine 200 mM (100×); Ref 25030-024; by Gibco by Life Technologies). Cultures were split every two-three days with a ratio of 1:10/1:15.

Mice

Mice were 7-8 week-old BALB/cJBomTac purchased from Taconic, Denmark. After arrival at the animal facility, mice were allowed to adjust to ambient conditions for at least 5 days before they were used for experiments. They were housed in Macrolon® type III cages in groups of ten under standardized conditions at 21.5±1.5° C. and 55±10% humidity. Standardized irradiated diet (PROVIMI KLIBA) and autoclaved tap water were provided ad libitum. Microchips implanted subcutaneously under isoflurane anesthesia were used to identify each mouse. Cage cards showing the study number, the animal number, the compound and dose level, the administration route as well as the schedule remained with the animals throughout the study.

Administration of Test Compounds

BIA-1 was suspended in 0.5% Natrosol and administered intragastrically using a gavage needle at an application volume of 10 mL/kg per mouse once daily.

The PD-1 antibody was diluted in PBS and injected intraperitoneally with a volume of 10 mL/kg per mouse twice weekly.

Monitoring Tumor Growth and Disease Progression

The tumor diameter was measured three times a week (Monday, Wednesday and Friday) with a caliper. The volume of each tumor [in mm³] was calculated according to the formula “tumor volume=length*diameter2*π/6”. To monitor side effects of treatment, mice were inspected daily for abnormalities and body weight was determined daily. Animals were sacrificed at the end of the study. Animals with necrotic tumors or tumor sizes exceeding 1500 mm³ were sacrificed early during the studies for ethical reasons.

Results

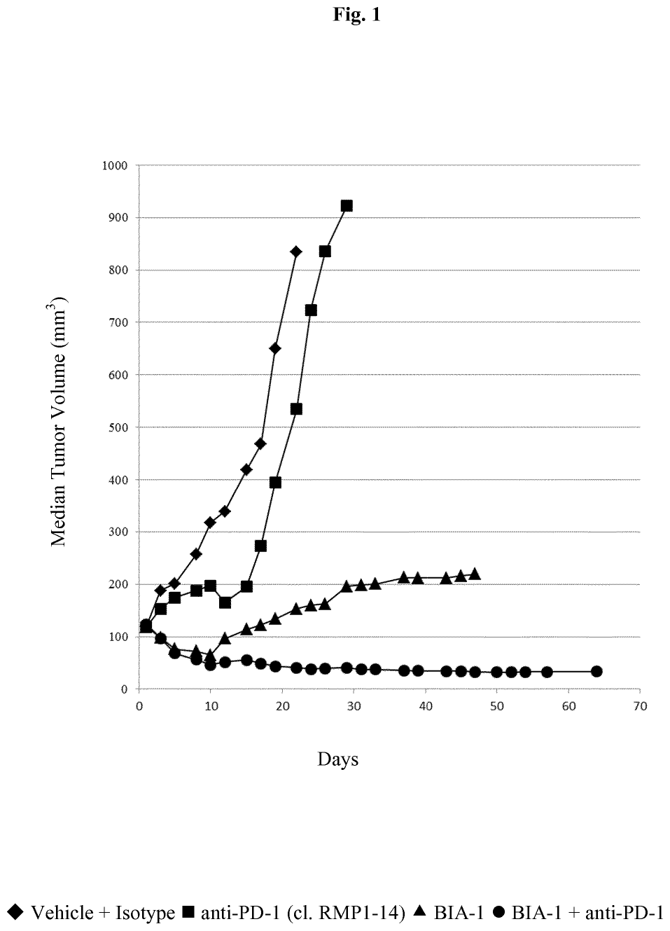

Treatment of ETM6 tumors with the SMAC mimetic BIA-1 as a single agent showed anti-tumor efficacy and was well tolerated. Treatment with the mouse tool antibody against PD-1 (RMP1-14) resulted in moderate tumor growth inhibition. Combination of the SMAC mimetic with the PD-1 antagonist resulted in increased efficacy when compared with single agent administrations, inducing tumor regressions in all mice. The results are shown in FIG. 1.

### Example 2

Anti-Tumor Activity of the Exemplary SMAC Mimetics BIA-1 and BIA-2 as Single Agents and in Combination with RMP1-14, a Mouse Tool Antibody to PD-1, in a Subcutaneous Syngeneic Mouse Model Derived from the Bladder Cancer Cell Line MBT-2 in C3H Mice.

The efficacy of the exemplary SMAC mimetics BIA-1 and BIA-2 was tested in a s.c. cell line derived syngeneic model of mouse bladder cancer (MBT-2) as single agents and in combination with RMP1-14, a mouse antibody to PD-1 (BioXCell # BE0146).

C3H mice were used in this study. Each mouse was inoculated subcutaneously at the right flank region with MBT-2 tumor cells (4×10⁵) in 0.1 mL of PBS mixed with matrigel (1:1) for tumor development. The treatments were started when the mean tumor size reached 83 mm³ on Day 7 after inoculation.

Eight tumor-bearing mice per group were treated daily per os with the SMAC mimetics, twice weekly i.p. with RMP1-14 or a combination of both compounds. Eight animals were used in the vehicle/isotype control-treated group. Animals for which the tumor size exceeded 1500 mm³ were euthanized prior to death. For animals which were killed based on tumor size (>1500 mm³), last value of the tumor volume was carried forward until the end of the study or until less than 70% of mice were still alive.

Cells

The MBT-2 cell line was maintained as monolayer culture in RPMI-1640 supplemented with 10% fetal bovine serum (FBS) at 37° C. in an atmosphere with 5% CO₂. The tumor cells were routinely subcultured 2× per week. The cells in an exponential growth phase were harvested and counted for tumor inoculation.

Mice

Mice were 7-8 week-old C3H purchased from Vital River Laboratory Animal Technology Co. (VR, Beijing, China).

The mice were kept in individually ventilated cage (IVC) systems at constant temperature and humidity with four animals in each cage. (−Temperature: 21˜25° C.,—Humidity: 59˜70%). The cages were made of polycarbonate. The size of each cage is 325 mm×210 mm×180 mm. The bedding material was corn cob (AWR Laboratory Animal Product Co., Ltd). The mouse diet was Co60 irradiation sterilized dry granule food (rodent growth and breeding diet, Jiangsu Province Synergistic Biological Engineering Co., LTD). Animals had free access during the entire study period. Water was reverseosmosis (RO) water, autoclaved before using. Animals had free access to sterile drinking water. The identification labels for each cage contained the following information: number of animals, sex, strain, receiving date, treatment, study number, group number, and the starting date of the treatment. Animal identification was done by ear coding (notch).

Administration of the Test Compound

The SMAC mimetics were suspended in 0.5% Natrosol and administered intragastrically using a gavage needle at an application volume of 10 mL/kg per mouse once daily.

The PD-1 antibody was diluted in PBS and injected intraperitoneally with a volume of 10 mL/kg per mouse twice weekly.

Monitoring Tumor Growth and Disease Progression

After tumor cell inoculation, the animals were checked daily for morbidity and mortality. At the time of routine monitoring, the animals were checked for any effects of tumor growth and treatments on normal behaviour such as mobility, visual estimation of food and water consumption, body weight gain/loss (body weights were measured thrice weekly), eye/hair matting and any other abnormal effect. Death and observed clinical signs were recorded on the basis of the numbers of animals within each subset.

Tumor volumes were measured twice weekly in two dimensions using a caliper, and the volume was expressed in mm³ using the formula: V=0.5 a×b2 where a and b are the long and short diameters of the tumor, respectively. Tumor weight was measured at study termination. The entire procedures of dosing as well as tumor and body weight measurement were conducted in a Laminar Flow Cabinet.

Results

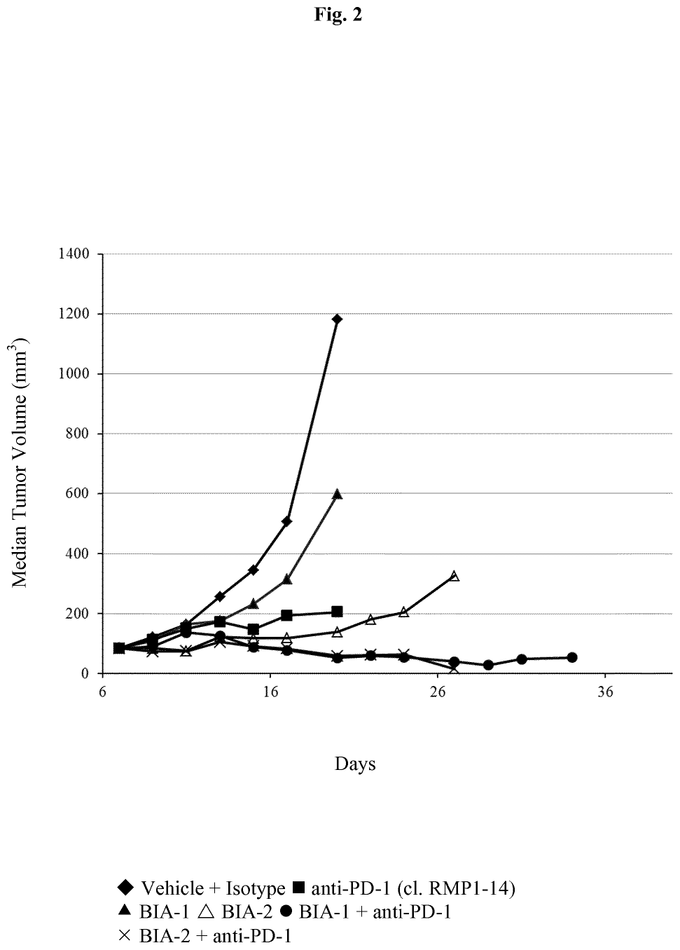

Treatment of MBT-2 tumors with either BIA-1 or BIA-2 monotherapies resulted in tumor growth inhibition. The combination of BIA-1 and BIA-2 with the PD-1 anatagonist RMP1-14 resulted in greater tumor growth inhibition than either monotherapy alone. The results are shown in FIG. 2.

### Example 3

Anti-Tumor Activity of the Exemplary SMAC Mimetics BIA-1 and BIA-2 as Single Agents and in Combination with RMP1-14, a Mouse Tool Antibody to PD-1, in the Vk12598 Multiple Myeloma Transplantable Model in C57BL/6J Mice.

The efficacy of the exemplary SMAC mimetics BIA-1 and BIA-2 was tested in a transplantable model of mouse multiple myeloma (Vk12598) as single agents and in combination with RMP1-14, a mouse antibody to PD-1 (BioXCell # BE0146).

C57BL/6J wild type mice were engrafted by i.v. injection with one million splenocytes from Vk*MYC derived Vk12598 tumor cells. Beginning four weeks post-transplant, recipient mice were bled weekly by tail grazing and serum protein electrophoresis (SPEP) and densitometric analysis were performed to measure M-spike levels and gamma/albumin ratio, as a measurement of tumor burden.

Mice with M-spike>7 g/L were randomized into vehicle or treatment groups, seven mice per treatment arm. Body weight was measured daily. SPEP was performed weekly beginning at day 0 and at day 7 and day 14 to measure M-spike levels as fraction of day 0 levels.

Administration of the Test Compound

The SMAC mimetics were suspended in 0.5% Natrosol and administered intragastrically using a gavage needle at an application volume of 10 mL/kg per mouse once daily.

The PD-1 antibody was diluted in PBS and injected intraperitoneally with a volume of 10 mL/kg per mouse twice weekly.

Results

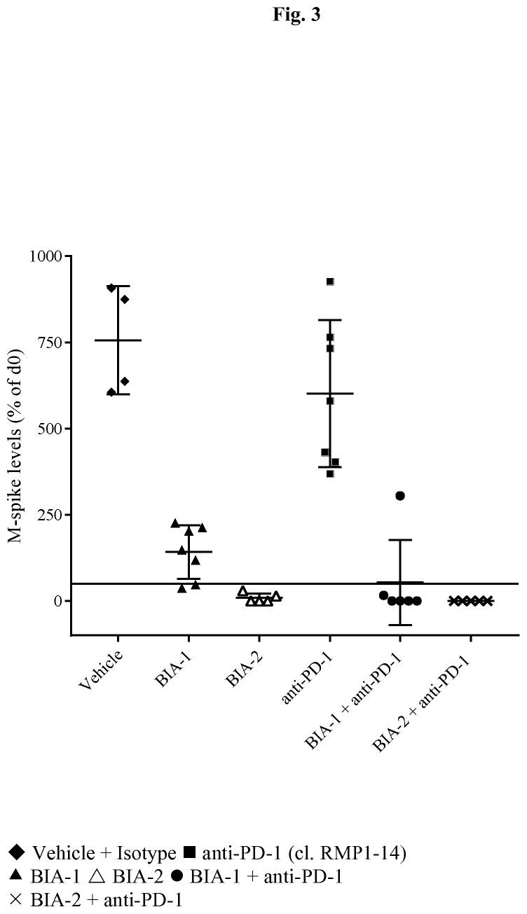

The anti-tumor response for BIA-1 and BIA-2 alone and in combination with the PD-1 antagonist, was evaluated by comparing the M-spike levels at 14 days post treatment to the day 0 levels. A response (>50% M-spike reduction) was noted in 2/7 BIA-1 treated mice, and in 7/7 BIA-2 treated mice. The combination of BIA-1 and BIA-2 resulted in 7/7 responses, respectively. No response was observed in the vehicle or anti-PD-1 treatment groups. The results are shown in FIG. 3.

### Example 4

Potentiating Activity of the Exemplary SMAC Mimetic BIA-1 on the Stimulation of Antigen-Specific T Cell Response by the Anti-PD1 MK3465

The exemplary SMAC mimetic BIA-1 and the SMAC mimetic LCL 161 were tested for their ability to potentiate the stimulation of INFγ production of Tetanus specific CD4 memory T cells induced by the anti-PD1 antibody MK3465 (Pembrolizumab).

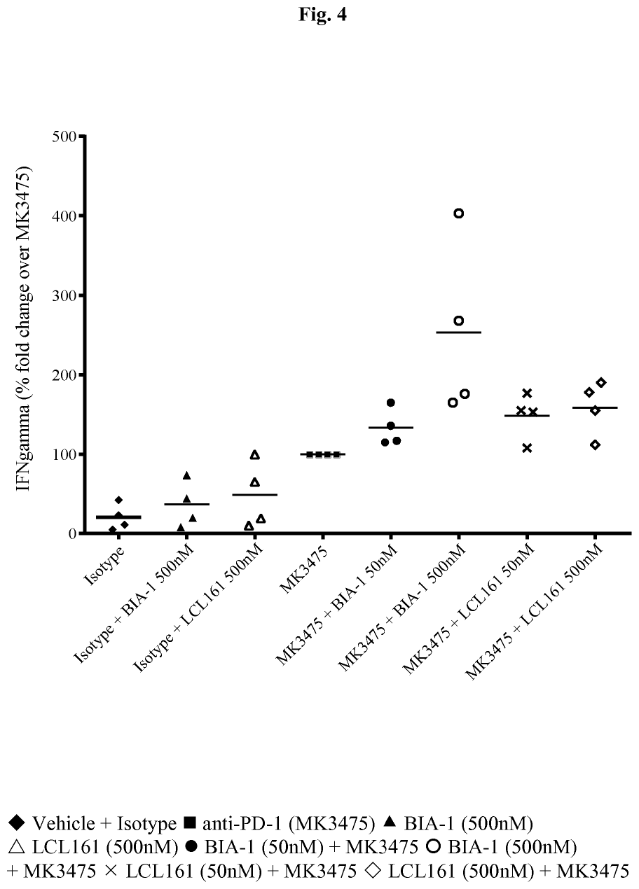

T cells from healthy donor PBMCs (n=4) were expanded in the presence of tetanus toxoid and co-cultured with autologous mature Dendritic Cells (DCs) loaded with tetanus toxoid for three days. The co-culture step was repeated a second time for five days in the presence of MK3465, BIA-1 (500 nM), LCL 161 (500 nM) or the combination of BIA-1 (50 nM and 500 nM)+MK3465 or LCL 161 (50 nM, 500 nM)+MK3465. At the end of the second co-culture step supernatants were analysed by ELISA for INFγ secretion. BIA-1 at 500 nM potentiates the ability of MK3465 to stimulate INFγ production of tetanus toxin specific CD4 memory T cells and results are shown in FIG. 4.

Examplary SMAC mimetic BIA-1 used for these experiments is one of the compounds disclosed in table 1.

1. A method of treating and/or preventing an oncological or hyperproliferative disease, said method comprising administering an SMAC mimetic in combination with a PD-1 antagonist to a patient in need thereof,
wherein the SMAC mimetic is selected from any one of the following compounds 1 to 26; 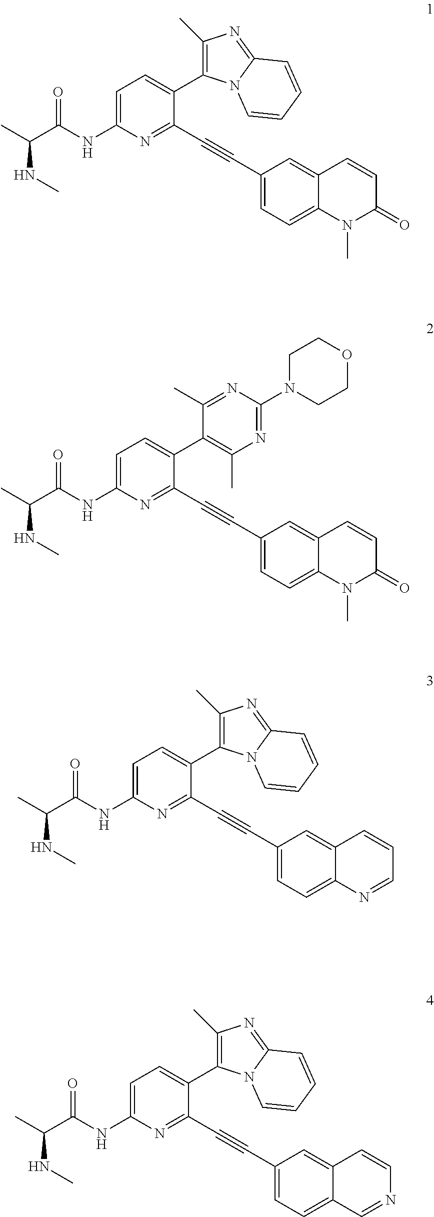 [SMILES: CN[C@@H](C)C(=O)Nc1ccc(-c2c(C)nc3ccccn23)c(C#Cc2ccc3c(ccc(=O)n3C)c2)n1 ; CN[C@@H](C)C(=O)Nc1ccc(-c2c(C)nc(N3CCOCC3)nc2C)c(C#Cc2ccc3c(ccc(=O)n3C)c2)n1 ; CN[C@@H](C)C(=O)Nc1ccc(-c2c(C)nc3ccccn23)c(C#Cc2ccc3ncccc3c2)n1 ; CN[C@@H](C)C(=O)Nc1ccc(-c2c(C)nc3ccccn23)c(C#Cc2ccc3cnccc3c2)n1] 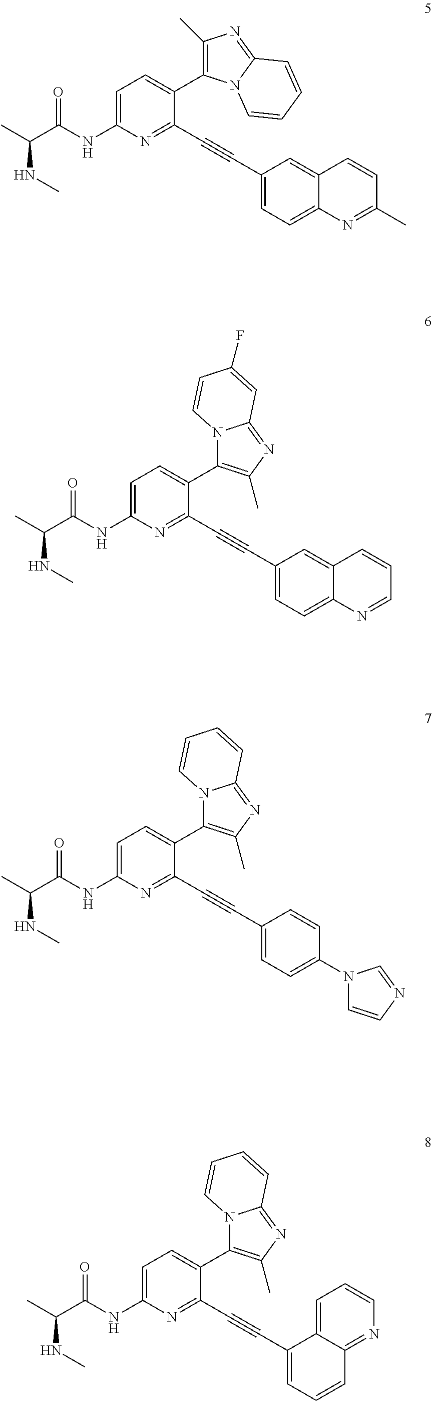 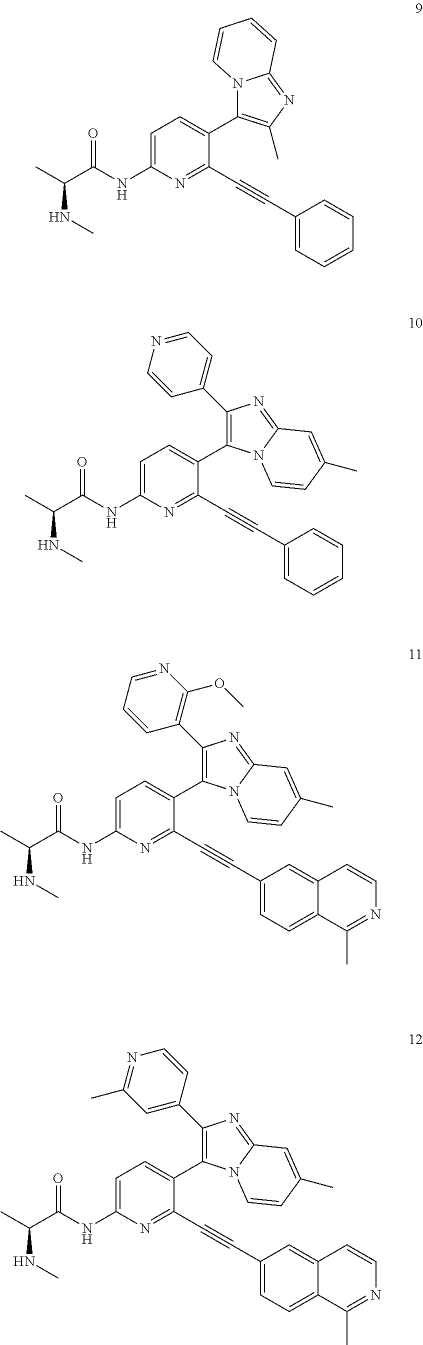 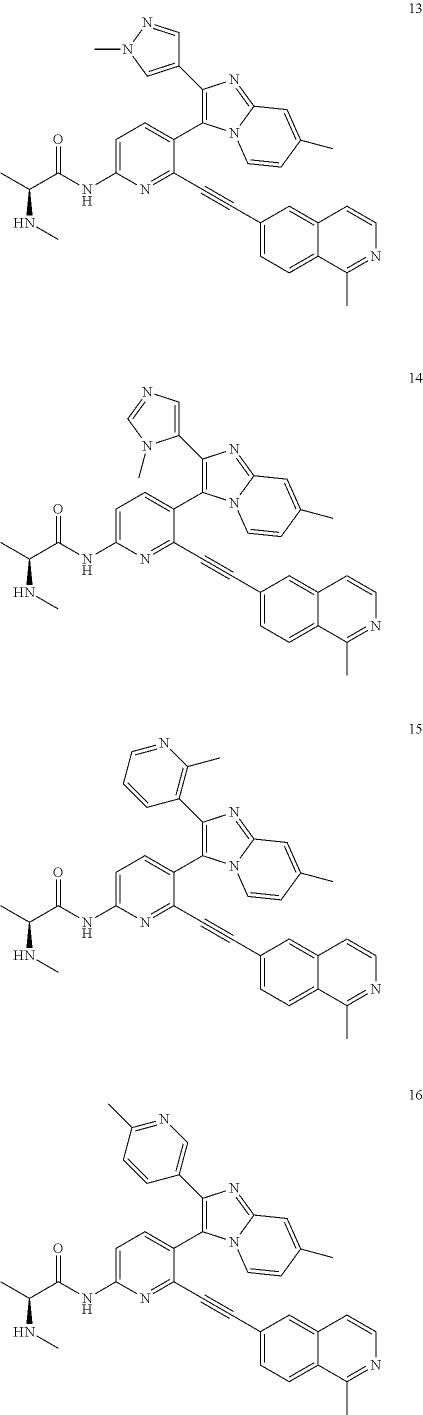 [SMILES: CN[C@@H](C)C(=O)Nc1ccc(-c2c(-c3cnn(C)c3)nc3cc(C)ccn23)c(C#Cc2ccc3c(C)nccc3c2)n1 ; CN[C@@H](C)C(=O)Nc1ccc(-c2c(-c3cncn3C)nc3cc(C)ccn23)c(C#Cc2ccc3c(C)nccc3c2)n1 ; CN[C@@H](C)C(=O)Nc1ccc(-c2c(-c3cccnc3C)nc3cc(C)ccn23)c(C#Cc2ccc3c(C)nccc3c2)n1 ; CN[C@@H](C)C(=O)Nc1ccc(-c2c(-c3ccc(C)nc3)nc3cc(C)ccn23)c(C#Cc2ccc3c(C)nccc3c2)n1] 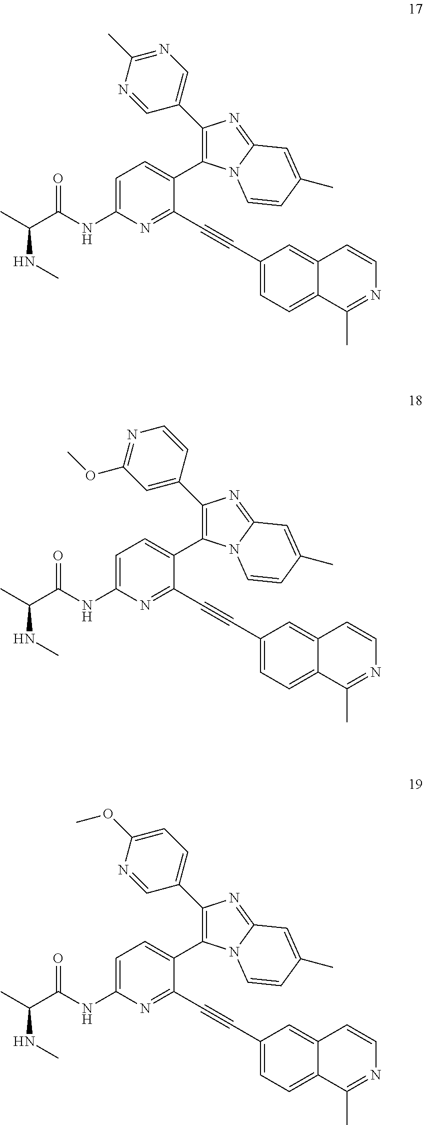 [SMILES: CN[C@@H](C)C(=O)Nc1ccc(-c2c(-c3cnc(C)nc3)nc3cc(C)ccn23)c(C#Cc2ccc3c(C)nccc3c2)n1 ; CN[C@@H](C)C(=O)Nc1ccc(-c2c(-c3ccnc(OC)c3)nc3cc(C)ccn23)c(C#Cc2ccc3c(C)nccc3c2)n1 ; CN[C@@H](C)C(=O)Nc1ccc(-c2c(-c3ccc(OC)nc3)nc3cc(C)ccn23)c(C#Cc2ccc3c(C)nccc3c2)n1] 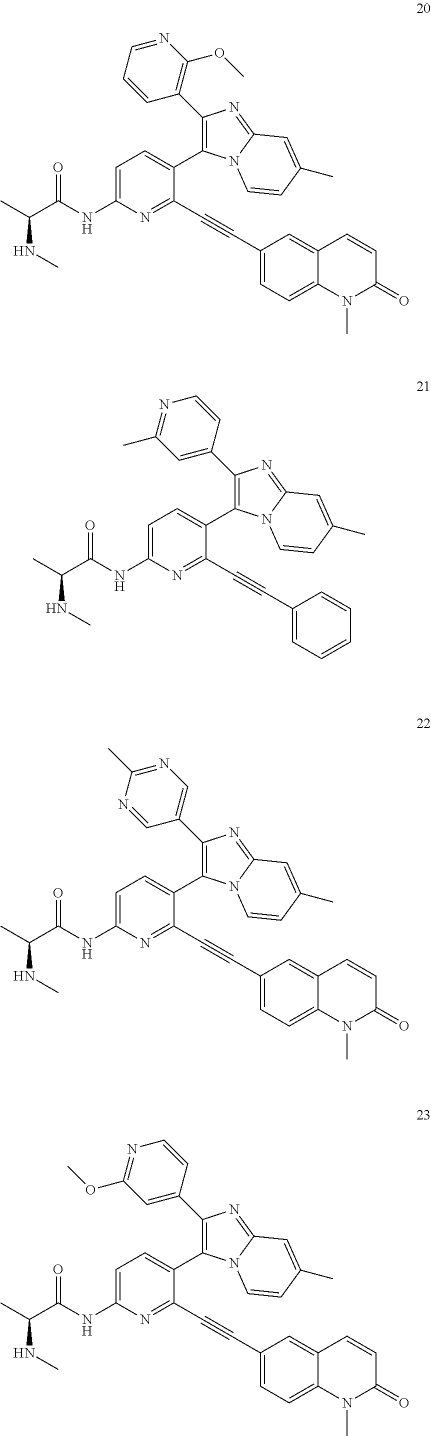 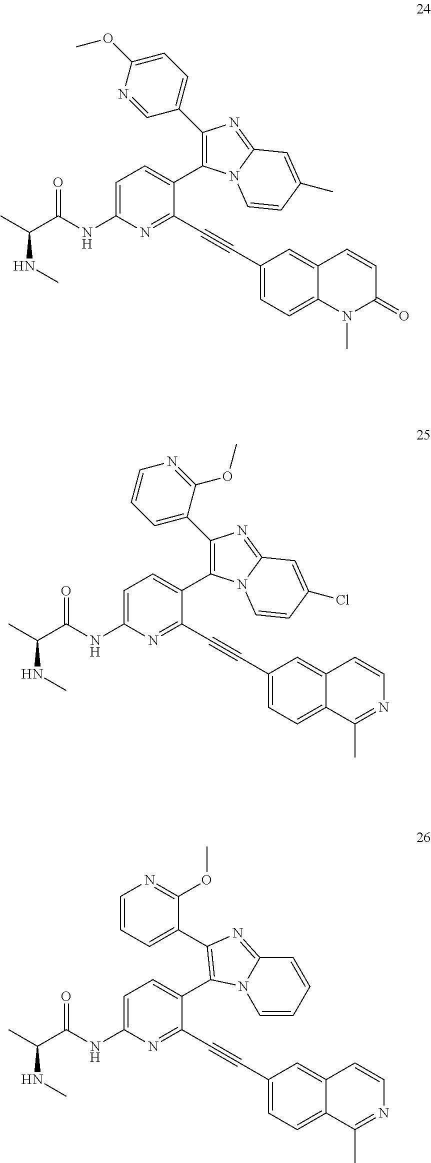 [SMILES: CN[C@@H](C)C(=O)Nc1ccc(-c2c(-c3ccc(OC)nc3)nc3cc(C)ccn23)c(C#Cc2ccc3c(ccc(=O)n3C)c2)n1 ; CN[C@@H](C)C(=O)Nc1ccc(-c2c(-c3cccnc3OC)nc3cc(Cl)ccn23)c(C#Cc2ccc3c(C)nccc3c2)n1 ; CN[C@@H](C)C(=O)Nc1ccc(-c2c(-c3cccnc3OC)nc3ccccn23)c(C#Cc2ccc3c(C)nccc3c2)n1]
or a pharmaceutically acceptable salt thereof; and
wherein the PD-1 antagonist is selected from the group consisting of pembrolizumab, nivolumab, pidilizumab, atezolizumab, avelumab, durvalumab, PDR-001, PD1-1, PD1-2, PD1-3, PD1-4 and PD1-5.

2. The method according to claim 1, wherein the SMAC mimetic is administered simultaneously, concurrently, sequentially, successively, alternately or separately with the PD-1 antagonist.

3. (canceled)

4. (canceled)

5. (canceled)

6. (canceled)

7. (canceled)

8. (canceled)

9. (canceled)

10. (canceled)

11. A pharmaceutical composition comprising:
a SMAC mimetic;
a PD-1 antagonist; and,
optionally, one or more pharmaceutically acceptable carriers, excipients and/or vehicles;
wherein the SMAC mimetic is selected from any one of compounds 1 to 26 according to claim 1 or a pharmaceutically acceptable salt of one of these compounds; and
wherein the PD-1 antagonist is selected from the group consisting of pembrolizumab, nivolumab, pidilizumab, atezolizumab, avelumab, durvalumab, PDR-001, PD1-1, PD1-2, PD1-3, PD1-4 and PD1-5.

12. (canceled)

13. A kit comprising:
a first pharmaceutical composition or dosage form comprising a SMAC mimetic and, optionally, one or more pharmaceutically acceptable carriers, excipients and/or vehicles;
a second pharmaceutical composition or dosage form comprising a PD-1 antagonist and, optionally, one or more pharmaceutically acceptable carriers, excipients and/or vehicles;
wherein the SMAC mimetic is selected from any one of compounds 1 to 26 according to claim 1 or a pharmaceutically acceptable salt of one of these compounds; and
wherein the PD-1 antagonist is selected from the group consisting of pembrolizumab, nivolumab, pidilizumab, atezolizumab, avelumab, durvalumab, PDR-001, PD1-1, PD1-2, PD1-3, PD1-4 and PD1-5.

14. (canceled)

15. (canceled)

16. The kit according to claim 13 further comprising:
a package insert comprising printed instructions for simultaneous, concurrent, sequential, successive, alternate or separate administration of the SMAC mimetic and PD-1 antagonist in the treatment and/or prevention of an oncological or hyperproliferative disease in a patient in need thereof.

17. The method according to claim 1, wherein the oncological disease to be treated is a cancer selected from the group consisting of lung cancer, multiple myeloma (MM) and breast cancer.

18. The method according to claim 17, wherein the cancer is non-small cell lung cancer (NSCLC).

19. The method according to claim 18, wherein the cancer is non-small cell lung cancer adenocarcinoma.

20. The method according to claim 17, wherein the cancer is triple-negative breast cancer (TNBC).


SEQUENCE LISTING (10 sequences)

SEQ ID NO: 1 - protein, 446 aa, Artificial Sequence
  HC of PD1-1
EVMLVESGGGLVQPGGSLRLSCTASGFTFSASAMSWVRQAPGKGLEWVAYISGGGGDTYYSSSVKGRFTISRDNAKNSLYLQMNSLRAEDTAVYYCARHSNVNYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 2 - protein, 218 aa, Artificial Sequence
  LC of PD1-1
EIVLTQSPATLSLSPGERATMSCRASENIDTSGISFMNWYQQKPGQAPKLLIYVASNQGSGIPARFSGSGSGTDFTLTISRLEPEDFAVYYCQQSKEVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 446 aa, Artificial Sequence
  HC of PD1-2
EVMLVESGGGLVQPGGSLRLSCTASGFTFSASAMSWVRQAPGKGLEWVAYISGGGGDTYYSSSVKGRFTISRDNAKNSLYLQMNSLRAEDTAVYYCARHSNPNYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 4 - protein, 218 aa, Artificial Sequence; identical to SEQ ID NO: 2
  LC of PD1-2

SEQ ID NO: 5 - protein, 446 aa, Artificial Sequence
  HC of PD1-3
EVMLVESGGGLVQPGGSLRLSCTASGFTFSKSAMSWVRQAPGKGLEWVAYISGGGGDTYYSSSVKGRFTISRDNAKNSLYLQMNSLRAEDTAVYYCARHSNVNYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 6 - protein, 218 aa, Artificial Sequence
  LC of PD1-3
EIVLTQSPATLSLSPGERATMSCRASENIDVSGISFMNWYQQKPGQAPKLLIYVASNQGSGIPARFSGSGSGTDFTLTISRLEPEDFAVYYCQQSKEVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 446 aa, Artificial Sequence; identical to SEQ ID NO: 5
  HC of PD1-4

SEQ ID NO: 8 - protein, 218 aa, Artificial Sequence; identical to SEQ ID NO: 6
  LC of PD1-4

SEQ ID NO: 9 - protein, 446 aa, Artificial Sequence; identical to SEQ ID NO: 5
  HC of PD1-5

SEQ ID NO: 10 - protein, 218 aa, Artificial Sequence; identical to SEQ ID NO: 6
  LC of PD1-5